(19) United States
(12) Patent Application Publication
(10) Pub. No.: US 2025/0262309 A1
(43) Pub. Date: Aug. 21, 2025

(54) PROTEOLYSIS TARGETING CHIMERA (PROTAC) COMPOSITIONS USING UBIQUITIN CONJUGATING ENZYME LIGANDS

(71) Applicant: BPGbio, Inc., Framingham, MA (US)

(72) Inventor: Vivek K. Vishnudas, Chelmsford, MA (US)

(21) Appl. No.: 19/078,822

(22) Filed: Mar. 13, 2025

Related U.S. Application Data
(63) Continuation of application No. 18/993,777, filed on Jan. 1, 0001.
(60) Provisional application No. 63/332,305, filed on Apr. 19, 2022.

(51) Int. Cl. A61K 47/55; A61P 35/00

(52) U.S. Cl. CPC A61K 47/55; A61P 35/00

(57) ABSTRACT

Provided herein are E2 binding PROTACS and uses thereof.

### RELATED APPLICATIONS

This application claims the benefit of priority to U.S. provisional application No. 63/332,305, filed Apr. 19, 2022, the entire contents of which are incorporated herein by reference.

### BACKGROUND

Proteolysis targeting chimeras (PROTACs) have become an appealing technology for modulating a protein of interest (POI). PROTACs are heterobifunctional small molecules with three chemical elements: a ligand binding to a target protein, a ligand binding to E3 ubiquitin ligase, and a linker for conjugating these two ligands. See e.g., Sun et al., Sig Transduct Target Ther 4, 64 (2019). Different from the competitive- and occupancy-driven process of traditional inhibitors, PROTACs are catalytic in their mode of action and have the potential to degrade the target pathogenic proteins and regulate the related signaling pathways. PROTACs targeting about 50 proteins, many of which are clinically validate drug targets, have been developed with several in clinical trials for the treatment of cancer and other diseases.

A problem with current PROTACs, however, is that they are confined to engaging E3 ubiquitin ligase, which is susceptible to a high degree of mutation. Recent studies have demonstrated that acquired resistance to the PROTACs is tied to mutations in core components of the E3 ligase. (Zhang et al., Mol Cancer Ther. 2019 July; 18 (7): 1302-1311. doi: 10.1158/1535-7163.MCT-18-1129. Epub 2019 May 7). Alternative approaches to traditional PROTAC technology are therefore needed.

### SUMMARY

It has now been found that ubiquitin conjugating enzyme (E2) (e.g., UBE2K) can serve as an effective target in the design and use of PROTACs. Unlike E3 ligases, E2s such as UBE2K have a very low frequency of mutation, therefore reducing the risk of PROTAC acquired resistance in clinical practice.

Provided herein, therefore, are compounds and compositions which function to recruit target proteins to E2 enzyme (e.g., UBE2K) for ubiquitination and subsequent degradation, as well as methods of using the same. In one aspect, the present disclosure provides PROTAC compounds having an E2 enzyme binding moiety, a target protein binding moiety, and a linker. In one aspect, the poly-ubiquitinating activity and/or increased processivity of the E2 enzyme is independent of an E3 ligase. In one aspect, the E2 enzyme binding moiety serves to stabilize an E2-E2 dimer (e.g., a homodimer or heterodimer).

Also provided are uses for a disclosed PROTAC for degrading a target protein of interest.

Further provided are uses for a disclosed PROTAC for treating a disease such as a cancer.

### BRIEF DESCRIPTION OF THE DRAWINGS

FIG. 1 shows the E2 binding capacity of PROTACS based on the present disclosure.

FIG. 2 illustrates the dose dependent degradation of BRD4 in Hela cells using E2 binding PROTACS based on the present disclosure.

### DETAILED DESCRIPTION

Provided herein are PROTAC compounds comprising an E2 binding moiety, a target protein binding moiety, and a linker which covalently attaches the E2 binding moiety to the target protein binding moiety.

As used herein, an “E2 binding moiety”, “E2 binder”, or “E2 ligand” refers to a chemical moiety that binds an E2 enzyme. E2 binding moieties, include, but are not limited to those which bind an E2 enzyme selected from UBE2A, UBE2B, UBE2C, UBE2D1, UBE2D2, UBE2D3, UBE2D4, UBE2E1, UBE2E2, UBE2E3, UBE2G1, UBE2G2, UBE2H, UBE2J1, UBE2J2, UBE2K, UBE2L3, UBE2N, UBE2NL, UBE20, UBE2Q1, UBE2Q2, UBE2QL, UBE2R1, UBE2R2, UBE2S, UBE2T, UBE2U, UBE2V1, UBE2V2, UBE2W, and BIRC6. In one aspect, the E2 enzyme to which the E2 ligand binds is UBE2K.

In one aspect, as part of a first chemical embodiment, the E2 binding moiety is a compound of the Formula I:

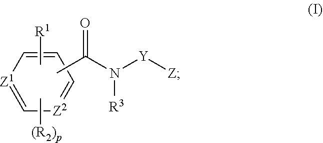

wherein
Z¹ and Z² are each independently N or CH;
R¹ is (C₁-C₆)alkyl, halo(C₁-C₆)alkyl, (C₁-C₆)alkoxy, halo(C₁-C₆)alkoxy, or —NR^{c}R^{d}, wherein two available hydrogen atoms on said halo(C₁-C₆)alkyl and halo(C₁-C₆)alkoxy may be taken together to which the carbon atoms they are attached to form a 3- to 6-membered cycloalkyl optionally substituted with 1 to 3 groups selected from halo, (C₁-C₆)alkyl, halo(C₁-C₆)alkyl, (C₁-C₆)alkoxy, and halo(C₁-C₆)alkoxy;
R² is CN, halo, OH, (C₁-C₆)alkyl, halo(C₁-C₆)alkyl, (C₁-C₆)alkoxy, or halo(C₁-C₆)alkoxy; or R¹ and R², when on adjacent carbon atoms, are taken together with the carbon atoms to which they are attached to form a 5- or 6-membered oxygen containing heterocyclyl optionally substituted with 1 to 3 groups selected from halo, (C₁-C₆)alkyl, and halo(C₁-C₆)alkyl;
R³ is hydrogen, (C₁-C₆)alkyl, or halo(C₁-C₆)alkyl;
Y is CH₂, —CHR₃, —CR^{a}R^{b}, S, or SO;
p is 0 or 1;
R^{a} and R^{b} are each independently halo, (C₁-C₆)alkyl, or halo(C₁-C₆)alkyl; or R^{a} and R^{b} together with the carbon atom they are bound for a 3- to 6-membered cycloalkyl or a 3- to 6-membered heterocyclyl, each of which are optionally substituted with 1 to 3 groups selected from halo, (C₁-C₆)alkyl, halo(C₁-C₆)alkyl, (C₁-C₆)alkoxy, halo(C₁-C₆)alkoxy, (C₁-C₆)alkylOH, (C₁-C₆)alkylO(C₁-C₆)alkyl, and OH;
R^{c} and R^{d} are each independently hydrogen (C₁-C₆)alkyl, halo(C₁-C₆)alkyl, (C₁-C₆)alkylO(C₁-C₆)alkyl, halo(C₁-C₆)alkylO(C₁-C₆)alkyl, (C₁-C₆)alkyl-O-halo(C₁-C₆)alkyl, halo(C₁-C₆)alkyl-O-halo(C₁-C₆)alkyl, or (C₁-C₆)alkylOH; or R^{e} and R^{d} together with the nitrogen atom they are bound form a 4- to 7-membered heterocyclyl optionally substituted with 1 to 3 groups selected from halo, (C₁-C₆)alkyl, halo(C₁-C₆)alkyl, (C₁-C₆)alkoxy, halo(C₁-C₆)alkoxy, and oxo;
Z is a tricyclic fused ring having the formula:

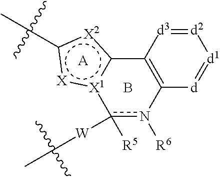

ring A is aromatic;
the wavy bond on ring A denotes the point of attachment to Y;
the wavy bond next to W denotes the point of attachment to Linker (L);
X, X¹, and X² are each, as valency permits, independently selected from —CR⁷, N, O, and S;
the dotted line in ring B represents a single or double bond;
W is NH, —N(C₁-C₆)alkyl), O, or S;
R⁵ is selected from hydrogen, (C₁-C₆)alkyl, halo(C₁-C₆)alkyl, (C₁-C₆)alkoxy, halo(C₁-C₆)alkoxy, —S(C₁-C₆)alkyl, —SH, OH, (C₃-C₆)cycloalkyl, (C₄-C₇)heterocyclyl, and —NR^{e}R^{f}, wherein said (C₃-C₆)cycloalkyl and (C₄-C₇)heterocyclyl are each optionally substituted with 1 to 3 groups selected from halo, (C₁-C₆)alkyl, halo(C₁-C₆)alkyl, (C₁-C₆)alkoxy, halo(C₁-C₆)alkoxy, and oxo;
R⁶ is hydrogen or (C₁-C₆)alkyl when the dotted line in ring B is a single bond, or R⁶ is absent when the dotted line in ring B is a double bond;
d, d₁, d₂ and d₃ are each independently selected from CR⁸ and N;
R^{e} and R^{f} are each independently hydrogen, (C₁-C₄)alkyl, (C₁-C₄)alkylNH (C₁-C₄)alkyl, (C₁-C₄)alkylN [(C₁-C₄)alkyl]₂, (C₁-C₄)alkylO(C₁-C₄)alkyl;
R⁷ is hydrogen or (C₁-C₆)alkyl; and
R⁸ is halogen, hydrogen, (C₁-C₆)alkyl, halo(C₁-C₆)alkyl, CN, or OH, wherein linker (L) and the target protein binding moiety are as defined herein.

When used in connection to describe a chemical group that may have multiple points of attachment, a hyphen (-) designates the point of attachment of that group to the variable to which is defined. For example, —NH(C₁-C₆)alkyl means that the point of attachment for this group is on the nitrogen atom.

The terms “halo” and “halogen” refer to an atom selected from fluorine (fluoro, —F), chlorine (chloro, —Cl), bromine (bromo, —Br), and iodine (iodo, —I).

The term “alkyl” when used alone or as part of a larger moiety, such as “haloalkyl”, means saturated straight-chain or branched monovalent hydrocarbon radical. Unless otherwise specified, an alkyl group typically has 1-4 carbon atoms, i.e., (C₁-C₄)alkyl.

“Alkoxy” means an alkyl radical attached through an oxygen linking atom, represented by -O-alkyl. For example, “(C₁-C₄)alkoxy” includes methoxy, ethoxy, proproxy, and butoxy.

The term “haloalkyl” includes mono, poly, and perhaloalkyl groups where the halogens are independently selected from fluorine, chlorine, bromine, and iodine.

“Haloalkoxy” is a haloalkyl group which is attached to another moiety via an oxygen atom such as, e.g., but are not limited to —OCHCF₂ or —OCF₃.

“Oxo” refers to the divalent function group ═O, i.e., an oxygen atom connected to another atom (typically carbon or sulfur) by a double bond.

The term “heteroaryl” refers to an aromatic ring of the specified size (e.g., 5-, 6-, 7-, 8-, or 9-membered ring) containing 1 to 4 heteroatoms independently selected from N, O, and S. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. Bi-cyclic heteroaryl include groups in which a monocyclic heteroaryl ring is fused to one or more aryl or heteroaryl rings. Nonlimiting examples include indolyl, imidazopyridinyl, benzooxazolyl, benzooxodiazolyl, indazolyl, benzimidazolyl, benzthiazolyl, pyrazolopyridinyl, thienopyridinyl, thienopyrimidinyl, indolizinyl, etc. When specified, optional substituents on a heteroaryl group may be present on any substitutable position.

The term “heterocyclyl” refers to a saturated or partially unsaturated monocyclic ring of the specified size (e.g., 3-, 4-, 5-, 6-, or 7-membered ring) containing 1 to 4 heteroatoms independently selected from N, O, and S. A heterocyclyl ring can be attached to its pendant group at any heteroatom or carbon atom that results in a stable structure. Examples of such saturated or partially unsaturated heterocyclic radicals include, without limitation, oxiranyl, thiiranyl, aziridinyl, tetrahydrofuranyl, tetrahydrothienyl, terahydropyranyl, pyrrolidinyl, pyridinonyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, oxetanyl, azetidinyl and tetrahydropyrimidinyl. When specified, optional substituents on a heterocyclyl group may be present on any substitutable position and, include, e.g., the position at which the heterocyclyl is attached.

The term “cycloalkyl” refers to a monocyclic hydrocarbon of the specified size (e.g., 3-, 4-, 5-, 6-, or 7-membered ring). Cycloalkyl groups include, without limitation, cyclopropyl, cyclobutyl, cyclopentyl, cyclopentenyl, cyclohexyl, cyclohexenyl, cycloheptyl, cycloheptenyl, and cyclooctyl. When specified, optional substituents on a cycloalkyl group may be present on any substitutable position and, include, e.g., the position at which the cycloalkyl is attached.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

The compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

In one aspect, as part of a second chemical embodiment, the E2 binding moiety is a compound of the Formula II:

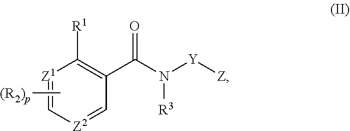

wherein the variables are as described above for Formula I and wherein linker (L) and the target protein binding moiety are as defined herein.

In one aspect, as part of a third chemical embodiment, W in the compound of Formula I or Formula II is S, wherein the variables are as described above for Formula I, wherein linker (L) and the target protein binding moiety are as defined herein.

In one aspect, as part of a fourth chemical embodiment, R³ in the compound of Formula I or Formula II is hydrogen, wherein the variables are as described above for Formula I or the third chemical embodiment, wherein linker (L) and the target protein binding moiety are as defined herein.

In one aspect, as part of a fifth chemical embodiment, Z¹ and Z² in the compound of Formula I or Formula II are each CH, wherein the variables are as described above for Formula I or the third or fourth chemical embodiment, wherein linker (L) and the target protein binding moiety are as defined herein.

In one aspect, as part of a sixth chemical embodiment, Y in the compound of Formula I or Formula II is CH₂, wherein the variables are as described above for Formula I or any one of the third to fifth chemical embodiments, wherein linker (L) and the target protein binding moiety are as defined herein.

In one aspect, as part of a seventh chemical embodiment, p in the compound of Formula I or Formula II is 0, wherein the variables are as described above for Formula I or any one of the third to sixth chemical embodiments, wherein linker (L) and the target protein binding moiety are as defined herein.

In one aspect, as part of an eighth chemical embodiment, R¹ in the compound of Formula I or Formula II is (C₁-C₆)alkyl, halo(C₁-C₆)alkyl, halo(C₁-C₆)alkoxy, (C₁-C₆)alkoxy, or —NR^{c}R^{d}; and R^{c} and R^{d} together with the nitrogen atom they are bound form a 5- to 6-membered heterocyclyl optionally substituted with 1 to 3 halo, wherein the variables are as described above for Formula I or any one of the third to seventh chemical embodiments, wherein linker (L) and the target protein binding moiety are as defined herein. Alternatively, as part of an eighth chemical embodiment, R¹ in the compound of Formula I or Formula II is (C₁-C₃)alkyl, halo(C₁-C₃)alkyl, halo(C₁-C₆)alkoxy, (C₁-C₃)alkoxy, or —NR^{c}R^{d}; and R^{e} and R^{d} together with the nitrogen atom they are bound form a 5- to 6-membered nitrogen containing heterocyclyl optionally substituted with 1 to 3 halo, wherein the variables are as described above for Formula I or any one of the third to seventh chemical embodiments, wherein linker (L) and the target protein binding moiety are as defined herein. In another alternative, as part of an eighth chemical embodiment, R¹ in the compound of Formula I or Formula II is OCF₃, OCHF₂, OCH₃, CH₃, pyrrolidinyl, or piperidinyl, wherein said pyrrolidinyl, or piperidinyl are optionally substituted with 1 to 3 halo, wherein the variables are as described above for Formula I or any one of the third to seventh chemical embodiments, wherein linker (L) and the target protein binding moiety are as defined herein.

In one aspect, as part of a ninth chemical embodiment, X in the compound of Formula I or Formula II is N, wherein the variables are as described above for Formula I or any one of the third to eighth chemical embodiments, wherein linker (L) and the target protein binding moiety are as defined herein.

In one aspect, as part of a tenth chemical embodiment, X¹ in the compound of Formula I or Formula II is CH or N, wherein the variables are as described above for Formula I or any one of the third to ninth chemical embodiments, wherein linker (L) and the target protein binding moiety are as defined herein.

In one aspect, as part of a eleventh chemical embodiment, X² in the compound of Formula I or Formula II is CH, N, S, or O, wherein the variables are as described above for Formula I or any one of the third to tenth chemical embodiments, wherein linker (L) and the target protein binding moiety are as defined herein.

In one aspect, as part of a twelfth chemical embodiment, X in the compound of Formula I or Formula II is N, X¹ is N, and X² is CH; X is N, X¹ is N, and X² is N; X is N, X¹ is CH, and X² is S; or X is N, X¹ is CH, and X² is O, wherein the variables are as described above for Formula I or any one of the third to eleventh chemical embodiments, wherein linker (L) and the target protein binding moiety are as defined herein.

In one aspect, as part of a thirteenth chemical embodiment, d in the compound of Formula I or Formula II is N or —CR⁸, wherein the variables are as described above for Formula I or any one of the third to twelfth chemical embodiments, wherein linker (L) and the target protein binding moiety are as defined herein.

In one aspect, as part of a fourteenth chemical embodiment, d² in the compound of Formula I or Formula II is N or —CR⁸, wherein the variables are as described above for Formula I or any one of the third to thirteenth chemical embodiments, wherein linker (L) and the target protein binding moiety are as defined herein.

In one aspect, as part of a fifteenth chemical embodiment, d¹ in the compound of Formula I or Formula II in the compound of Formula I or Formula II is —CR⁸, wherein the variables are as described above for Formula I or any one of the third to fourteenth chemical embodiments, wherein linker (L) and the target protein binding moiety are as defined herein.

In one aspect, as part of a sixteenth chemical embodiment, d³ in the compound of Formula I or Formula II is —CR⁸, wherein the variables are as described above for Formula I or any one of the third to fifteenth chemical embodiments, wherein linker (L) and the target protein binding moiety are as defined herein.

In one aspect, as part of a seventeenth chemical embodiment, Z in the compound of Formula I or Formula II is selected from

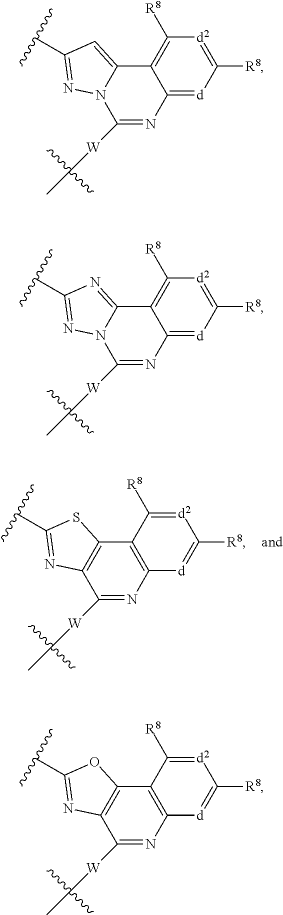

wherein the variables are as described above for Formula I or any one of the third to sixteenth chemical embodiments, wherein linker (L) and the target protein binding moiety are as defined herein. Alternatively, as part of a seventeenth chemical embodiment, Z in the compound of Formula I or Formula II is selected from

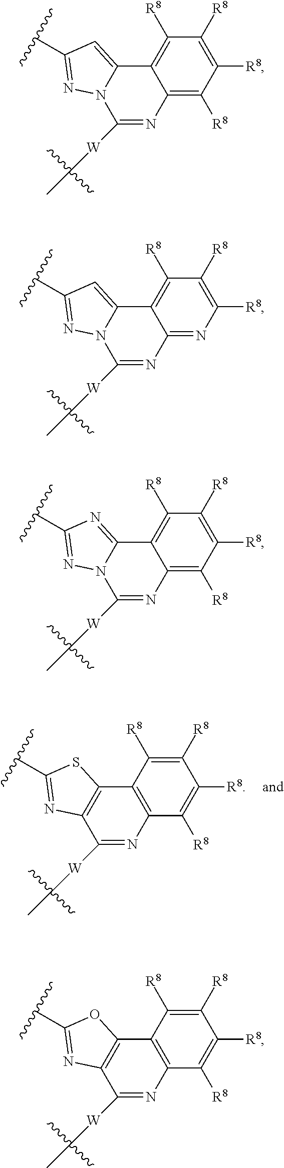

wherein the variables are as described above for Formula I or any one of the third to sixteenth chemical embodiments, wherein linker (L) and the target protein binding moiety are as defined herein.

In one aspect, as part of an eighteenth chemical embodiment, R⁸ in the compound of Formula I or Formula II is hydrogen or halo, wherein the variables are as described above for Formula I or any one of the third to seventeenth chemical embodiments, wherein linker (L) and the target protein binding moiety are as defined herein. Alternatively, as part of an eighteenth chemical embodiment, R⁸ in the compound of Formula I or Formula II is hydrogen, wherein the variables are as described above for Formula I or any one of the third to seventeenth chemical embodiments, wherein linker (L) and the target protein binding moiety are as defined herein.

In one aspect, as part of a nineteenth chemical embodiment, R⁵ in the compound of Formula I or Formula II is hydrogen, wherein the variables are as described above for Formula I or any one of the third to eighteenth chemical embodiments, wherein linker (L) and the target protein binding moiety are as defined herein.

In one aspect, as part of a twentieth chemical embodiment, R⁶ in the compound of Formula I or Formula II is selected from hydrogen and (C₁-C₄)alkyl, wherein the variables are as described above for Formula I or any one of the third to nineteenth chemical embodiments, wherein linker (L) and the target protein binding moiety are as defined herein. Alternatively, as part of a twentieth chemical embodiment, R⁶ in the compound of Formula I or Formula II is selected from hydrogen and methyl, wherein the variables are as described above for Formula I or any one of the third to nineteenth chemical embodiments, wherein linker (L) and the target protein binding moiety are as defined herein.

In one aspect, as part of a twenty-first chemical embodiment, the E2 binding moiety is a compound of the Formula III:

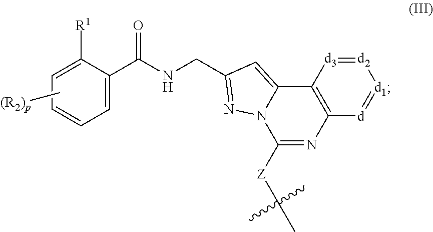

wherein d, d₁, d₂ and d₃ are each CR⁸ and wherein the variables are as described above for Formula I, and wherein linker (L) and the target protein binding moiety are as defined herein.

In one aspect, as part of a twenty-second chemical embodiment, p in the compound of Formula III is 0, wherein the variables are as described above for Formula I, and wherein linker (L) and the target protein binding moiety are as defined herein.

In one aspect, as part of a twenty-third chemical embodiment, the E2 binding moiety is of the structure;

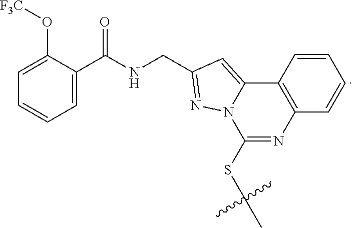 [SMILES: *Sc1nc2ccccc2c2cc(CNC(=O)c3ccccc3OC(F)(F)F)nn12]

In one aspect, as part of a twenty-third chemical embodiment, Z in the compound of Formula III is S, wherein the variables are as described above for Formula I or the twenty-second embodiment, and wherein linker (L) and the target protein binding moiety are as defined herein.

In one aspect, as part of a twenty-fourth chemical embodiment, the E2 binding moiety is selected from one disclosed in U.S. Provisional 62/223,626.

As used herein, a “target protein binding moiety” refers to a chemical moiety which bind a protein of interest (POI). POIs for the disclosed compounds include, but are not limited to, BRD4, STAT3, BCL2, and WRN. In one aspect, the POI is BRD4.

In one aspect, the target protein binding moiety disclosed herein (e.g., in any one of the first to twenty-fourth embodiments above) is of the structure:

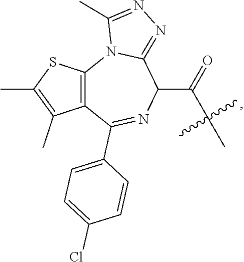 [SMILES: *C(=O)C1N=C(c2ccc(Cl)cc2)c2c(sc(C)c2C)-n2c(C)nnc21]

wherein the wavy bond denotes the point of attachment to Linker (L).

The term “linker” refers to a chemical moiety which covalently attaches the E2 binding moiety to the target protein binding moiety. In one aspect, the linker is uncleavable in vivo. In one aspect, the linker comprises optimal spatial and chemical properties to effectuate optimal therapeutic activity. In one aspect, the linker does not interfere with the ability of the E2 binding moiety and/or the target protein binding moiety to bind their respective targets. In one aspect, the linker comprises an optionally substituted straight or branched alkyl group which is optionally interrupted by one or more heteroatoms selected from O, N, and S. In one aspect, the linker is a straight or branched alkyl group substituted with one or more oxo groups (═O) and is interrupted by one more heteroatoms selected from O and N.

In one aspect, Linker (L) as disclosed herein (e.g., in any one of the first to twenty-fourth embodiments above) is of the following structure:

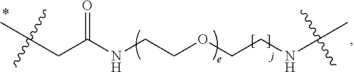

wherein the asterisk (*) denotes the point of attachment to the E2 binding moiety; e is an integer from 0 to 4 (e.g., 0, 1, or 2); and j is an integer from 0 to 6 (e.g., 1). Alternatively, Linker (L) as disclosed herein (e.g., in any one of the first to twenty-fourth embodiments above) is selected from one of the following structures:

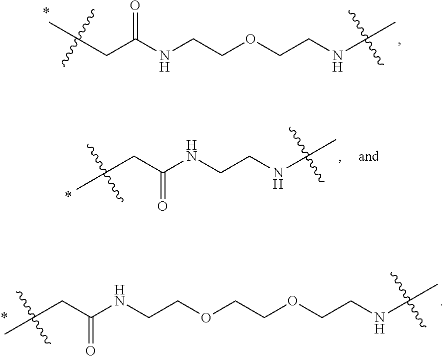 [SMILES: *CC(=O)NCCOCCN* ; *CC(=O)NCCN* ; *CC(=O)NCCOCCOCCN*]

Additional compounds are disclosed in the Exemplification and are included in the present disclosure. Pharmaceutically acceptable salts thereof as well as the neutral forms are included,

### Uses, Formulation and Administration

One or more compounds described herein may be present as part of a pharmaceutical composition. Therefore, in one aspect, provided are pharmaceutical compositions comprising a disclosed compound, or a pharmaceutically acceptable salt thereof; and a pharmaceutically acceptable carrier.

The term “pharmaceutically acceptable” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

The compounds and compositions described herein are generally useful for modulating the activity of the target protein (e.g., STAT3, BCL2, WRN, or BRD4). In some aspects, the compounds, pharmaceutical acceptable salts, and pharmaceutical compositions described herein degrade the target protein (e.g., STAT3, BCL2,WRN, or BRD4).

Thus, provided herein are methods of treating a disease responsive to the degradation of a target protein (e.g., STAT3, BCL2, WRN, or BRD4) in a subject, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof.

Also provided is the use of a compound described herein, or a pharmaceutically acceptable salt thereof, or a pharmaceutical composition comprising a disclosed compound or pharmaceutically acceptable salt thereof for treating a disease responsive to the degradation of a target protein (e.g., STAT3, BCL2, WRN, or BRD4).

Also provided herein are methods of degrading a target protein (e.g., STAT3, BCL2,WRN, or BRD4) in a cell, comprising introducing into the cell a compound comprising an E2 binding moiety, a target protein binding moiety (i.e., binding moiety that binds to the target protein to be degraded), and a linker which covalently attaches the E2 binding moiety to the target protein binding moiety, thereby degrading the target protein in the cell.

Also provided herein are methods of degrading a target protein (e.g., STAT3, BCL2,WRN, or BRD4) in a subject, comprising administering to the subject an effective amount of a compound comprising an E2 binding moiety, a target protein binding moiety (i.e., binding moiety that binds to the target protein to be degraded), and a linker which covalently attaches the E2 binding moiety to the target protein binding moiety, or a pharmaceutically acceptable salt thereof, or a pharmaceutical composition comprising the compound or pharmaceutically acceptable salt thereof, thereby degrading the target protein in the subject.

In addition, provided herein are methods of treating a disease responsive to the degradation of a target protein (e.g., STAT3, BCL2, WRN, or BRD4) in a subject, comprising administering to a subject in need thereof, a therapeutically effective amount of an effective amount of a compound comprising an E2 binding moiety, a target protein binding moiety (i.e., binding moiety that binds to the target protein which, when degraded, treats the disease), and a linker which covalently attaches the E2 binding moiety to the target protein binding moiety, or a pharmaceutically acceptable salt thereof, or a pharmaceutical composition comprising the compound or pharmaceutically acceptable salt thereof, thereby treating the disease.

Also provided is the use of a compound described herein, or a pharmaceutically acceptable salt thereof, or a pharmaceutical composition comprising a disclosed compound or pharmaceutically acceptable salt thereof, for the manufacture of a medicament for treating a disease responsive to the degradation of a target protein (e.g., STAT3, BCL2, WRN, or BRD4).

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “effective amount” or “therapeutically effective amount” refers to an amount of a compound described herein that will elicit a biological or medical response of a subject e.g., a dosage of between 0.01-100 mg/kg body weight/day.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

In some aspects, the disease responsive to the degradation of a target protein include, but are not limited to, cancer and other proliferative disorders, inflammatory diseases, sepsis, autoimmune disease, and viral infection.

In some aspects, the disease to be treated by the present methods is a cancer. Examples of cancers treated using the compounds and methods described herein include, but are not limited to, adrenal cancer, acinic cell carcinoma, acoustic neuroma, acral lentigious melanoma, acrospiroma, acute eosinophilic leukemia, acute erythroid leukemia, acute lymphoblastic leukemia, acute megakaryoblastic leukemia, acute monocytic leukemia, actue promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogeous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, preimary peritoneal cancer, prostate cancer, pancreatic cancer, pharyngeal cancer, pseudomyxoma periotonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor.

In some aspects, the disease to be treated by the present methods is inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenia states, Goodpasture's syndrome, athersclerosis, Addison's disease, Parkinson's disease, Alzheimer's disease, Type I diabetes, septic shock, systemic lupus erythematosus (SLE), rheumatoid arthritis, psoriatic arthritis, juvenile arthritis, osteoarthritis, chronic idiopathic thrombocytopenia purpura, Waldenstrom macroglobulinemia, myasthenia gravis, Hashimoto's thyroiditis, atopic dermatitis, degenerative joint disease, vitiligo, autoimmune hypopituatarism, Guillain-Barre syndrome, Behcet's disease, scleracierma, mycosis fungoides, acute inflammatory responses (such as acute respiratory distress syndrome and ischemia/reperfusion injury), and Graves' disease.

### EXEMPLIFICATION

The representative examples that follow are intended to help illustrate the present disclosure, and are not intended to, nor should they be construed to, limit the scope of the invention. General starting materials used were obtained from commercial sources or prepared in other examples, unless otherwise noted.

### Abbreviations

| | |
|---|---|
| AcCl | Acetyl chloride |
| CAN | Acetonitrile |
| DCM | Dichloromethane |
| DIPEA | Diisopropylethylamine |
| DMF | N,N-Dimethylformamide |
| EDC•HCl | 1-Ethyl-3-(3′-dimethylaminopropyl)carbodiimide•HCl |
| EtOAc | Ethyl acetate |
| h | hour (s) |
| HATU | 1-[Bis(dimethylamino)methylene]-1H-1,2,3-triazolo[4,5- |
| | b]pyridinium 3-oxid hexafluorophosphate |
| HOBt | Hydroxybenzotriazole |
| Psi | Pound per square inch |
| RT | Room temperature |
| TEA | Triethylamine |
| TFA | Trifluoroacetic acid |
| THF | Tetrahydrofuran |

### Synthesis of N-((5-thioxo-5,6-dihydropyrazolo[1,5-c]quinazolin-2-yl)methyl)-2-(trifluoromethoxy)benzamide (Compound 1)

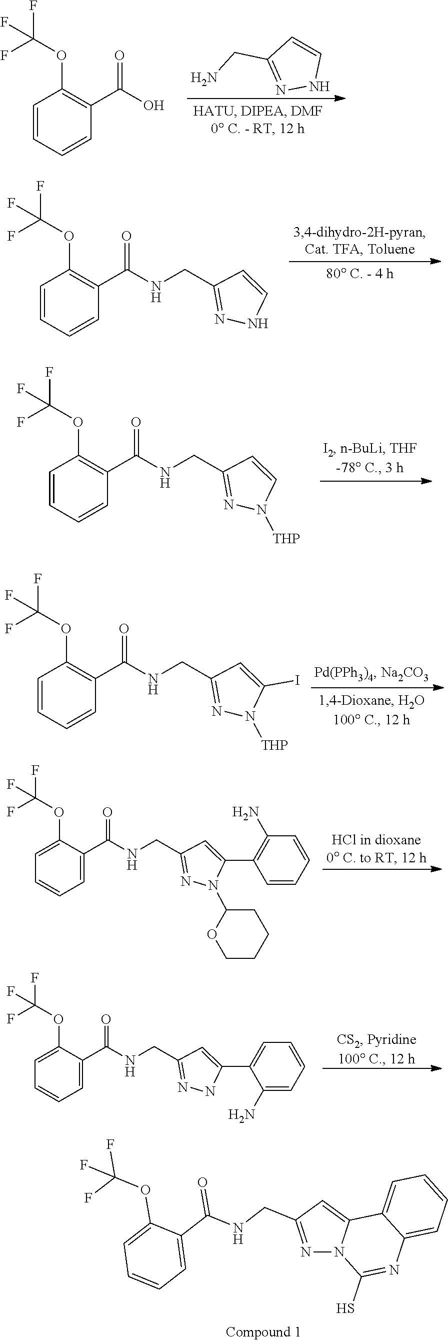

Step 1: Synthesis of N-((1H-pyrazol-3-yl)methyl)-2-(trifluoro methoxy)benzamide.

To a stirred solution of 2-(trifluoro methoxy)benzoic acid (1) (25 g, 0.121 mmol) in DMF (250 mL) at 0° C. was added HATU (46.1 g, 0.121), followed by (2H-pyrazol-3-yl) methanamine (11.7 g, 0.1213) and DIPEA (39.1 g, 0.303 mmol). Then the reaction was stirred at room temperature for 12 h. The mixture was then diluted with water (2.5L) and extracted with EtOAc (2×500 mL). The combined organic layer was washed once with H₂O (250 mL), saturated NaHCO₃ solution (250 mL), and finally saturated NaCl solution (250 mL). The organic layer was dried over Na₂SO₄ and concentrated to obtain the crude product. It was then purified by flash column chromatography (eluent: 70% EtOAc/Pet ether) to afford N-((1H-pyrazol-3-yl)methyl)-2-(trifluoro methoxy)benzamide (18.3 g, 53.0%) as off-white solid. ¹H NMR (400 MHZ, DMSO) δ 12.69-12.58 (m, 1H), 8.92-8.82 (m, 1H), 7.76-7.50 (m, 3H), 7.49-7.27 (m, 2H), 6.15 (d, J=12.1 Hz, 1H), 4.42 (t, J=11.1 Hz, 2H). LC-MS m/z (M+H): 286.1.

Step 2: Synthesis of N-((1-(tetrahydro-2H-pyran-2-yl)-1H-pyrazol-3-yl)methyl)-2˜ (trifluoro methoxy)benzamide.

To a stirred solution of N-((1H-pyrazol-3-yl)methyl)-2-(trifluoro methoxy)benzamide (18.3 g, 64.15 mmol) in toluene (400 mL) at room temperature was added 3,4-dihydro-2H-pyran (5.39 g, 64.1 mmol). Then the mixture was heated at 80° C. for 4 h and concentrated. The residue was diluted with EtOAc (250 mL), washed once with saturated NaHCO₃ solution (100 mL) and H₂O (100 mL). The organic layer was dried over Na₂SO₄ and concentrated. The crude product obtained was triturated with petroleum ether (200 mL) and stirred for 12h, where the solid precipitated was filtered, dried under vacuum to afford N-((1-(tetrahydro-2H-pyran-2-yl)-1H-pyrazol-3-yl)methyl)-2-(trifluoro methoxy)benzamide (12.57 g, 51.5%) as off-white solid. JH NMR (400 MHZ, DMSO) δ 8.87 (s, 1H), 7.80 (d, J=2.2 Hz, 1H), 7.57 (t, J=7.0 Hz, 2H), 7.46-7.38 (m, 2H), 6.20 (d, J=2.2 Hz, 1H), 5.32 (d, J=10.3 Hz, 1H), 4.38 (d, J=5.9 Hz, 2H), 3.90 (d, J=11.0 Hz, 1H), 3.67-3.52 (m, 1H), 2.07 (dd, J=24.7, 11.0 Hz, 1H), 1.98-1.80 (m, 2H), 1.65 (s, 1H), 1.51 (d, J=3.5 Hz, 2H); LC-MS m/z (M+H): 370.1.

Step 3: Synthesis of N-((5-iodo-1-(tetrahydro-2H-pyran-2-yl)-1H-pyrazol-3-yl)methyl)-2-(trifluoro methoxy)benzamide.

To a stirred solution of N-((1-(tetrahydro-2H-pyran-2-yl)-1H-pyrazol-3-yl)methyl)-2-(trifluoro methoxy)benzamide (18.2 g, 49.30 mmol) in dry THF (200 mL) at −78° C. was added 1.6M n-butyl lithium in hexane (6.31 g, 98.61 mmol) over a period of 10 min. Then the mixture was stirred at same temperature for 1h, followed by the addition of iodine (13.76 g, 54.2 mmol) in dry THF (200 mL) over a period of 15 min. After the complete addition of iodine, the reaction mixture was slowly allowed to warm up to −20° C. over a period of 45 min. The reaction was then quenched with saturated NaHSO₃ solution (200 mL) and extracted with EtOAc (2×150 mL). The combined organic layer was dried over Na₂SO₄, concentrated on rotavapor to get the crude compound. The crude compound was purified by flash column chromatography (eluent: 20% EtOAc+Pet ether) to N-((5-iodo-1-(tetrahydro-2H-pyran-2-yl)-1H-pyrazol-3-yl)methyl)-2-(trifluoro methoxy)benzamide (12.57 g, 51.5%) as off-white solid. ¹H NMR (400 MHZ, DMSO) δ 8.91 (t, J=5.6 Hz, 1H), 7.63-7.53 (m, 2H), 7.46-7.41 (m, 2H), 6.43 (s, 1H), 5.33 (d, J=9.8 Hz, 1H), 4.36 (d, J=5.6 Hz, 2H), 3.90 (d, J=10.9 Hz, 1H), 3.61-3.58 (m, 1H), 3.56-3.53 (m, 1H), 2.31-2.22 (m, 1H), 1.97 (d, J=12.3 Hz, 1H), 1.83 (d, J=12.1 Hz, 1H), 1.67 (s, 1H), 1.50 (s, 2H); LC-MS m/z (M+H): 396.0.

Step 4: Synthesis of N-((5-(2-aminophenyl)-1-(tetrahydro-2H-pyran-2-yl)-1H-pyrazol-3-yl)methyl)-2-(trifluoromethoxy)benzamide.

To a stirred solution of N-((5-iodo-1-(tetrahydro-2H-pyran-2-yl)-1H-pyrazol-3-yl)methyl)-2-(trifluoro methoxy)benzamide (6.64 g, 13.40 mmol) and (2-aminophenyl) boronic acid (2.20 g, 16.08 mmol) in 1,4-dioxane-water (61 mL+19 mL) was added Na₂CO₃ (3.55 g, 33.49 mmol). Then the mixture was degassed with argon for 10 min and to it was added tetrakis(triphenylphosphine) palladium (0) (1.54 g, 1.33 mmol). The resultant mixture was heated at 100° C. for 12 h, then diluted with H₂O (15 mL) and extracted with EtOAc (2×50 mL). The combined organic layer dried over Na₂SO₄ and concentrated. The crude product obtained was purified by flash column chromatography (eluent: 30% EtOAc+Hexane) to afford N-((5-(2-aminophenyl)-1-(tetrahydro-2H-pyran-2-yl)-1H-pyrazol-3-yl)methyl)-2-(trifluoromethoxy)benzamide as yellow gummy solid (4.6 g, 74.5%). LC-MS m/z (M−H): 461.11.

Step 5: Synthesis of N-((S-(2-aminophenyl)-1H-pyrazol-3-yl)methyl)-2-(trifluoromethoxy)benzamide.

To a stirred solution of N-((5-(2-aminophenyl)-1-(tetrahydro-2H-pyran-2-yl)-1H-pyrazol-3-yl)methyl)-2-(trifluoromethoxy)benzamide (3.70 g, 8.03 mmol) in DCM (20 mL) at 0° C. was added HCl in 1,4-dioxane (4M, 30 mL). Then the mixture was warmed up and stirred at room temperature for 12h. It was then concentrated and further co-distilled with DCM (2×25 mL) to get the crude compound. The crude compound was diluted with H₂O (25 mL), basified with saturated NaHCO₃ solution (25 mL) and extracted with EtOAc (2×25 mL). The combined organic layer was dried over Na₂SO₄ and concentrated to afford N-((5-(2-aminophenyl)-1H-pyrazol-3-yl)methyl)-2-(trifluoromethoxy)benzamide as yellow gummy solid (4.75 g); LC-MS m/z (M−H): 377.19.

Step 6: Synthesis of N-((S-thioxo-5,6-dihydropyrazolo[1,5-c]quinazolin-2-yl)methyl)-2-(trifluoromethoxy)benzamide (Compound 1):

To a stirred solution of N-((5-(2-aminophenyl)-1H-pyrazol-3-yl)methyl)-2-(trifluoromethoxy)benzamide (4.75 g, 12.62 mmol) in pyridine (110 mL) and H₂O (30 mL) was added carbon disulfide (110 mL) at room temperature. Then the reaction mixture heated at 100° C. for 12h. It was then diluted with cold H₂O (500 mL), the solid precipitated was filtered and air-dried to obtain the crude compound. It was then purified by prep-HPLC to afford N-((5-thioxo-5,6-dihydropyrazolo[1,5-c]quinazolin-2-yl)methyl)-2-(trifluoromethoxy)benzamide as off-white solid (1.6 g, 30.30%). ¹H NMR (400 MHZ, DMSO) δ 13.52 (s, 1H), 9.17 (t, J=5.8 Hz, 1H), 8.07 (d, J=7.8 Hz, 1H), 7.76-7.65 (m, 1H), 7.62-7.56 (m, 3H), 7.51-7.36 (m, 3H), 7.23 (s, 1H), 4.64 (d, J=5.9 Hz, 2H); LC-MS m/z (M+H): 419.26.

### Synthesis(S)-N-((5-((2-(2-(4-(4-chlorophenyl)-2,3,9-trimethyl-6H-thieno[3,2-f][1,2,4]triaz olo[4,3-a][1,4]diazepin-6-yl)acetamido)ethyl)thio)pyrazolo[1,5-c]quinazolin-2-yl)methyl)-2-(trifluoro methoxy)benzamide (Compound 2)

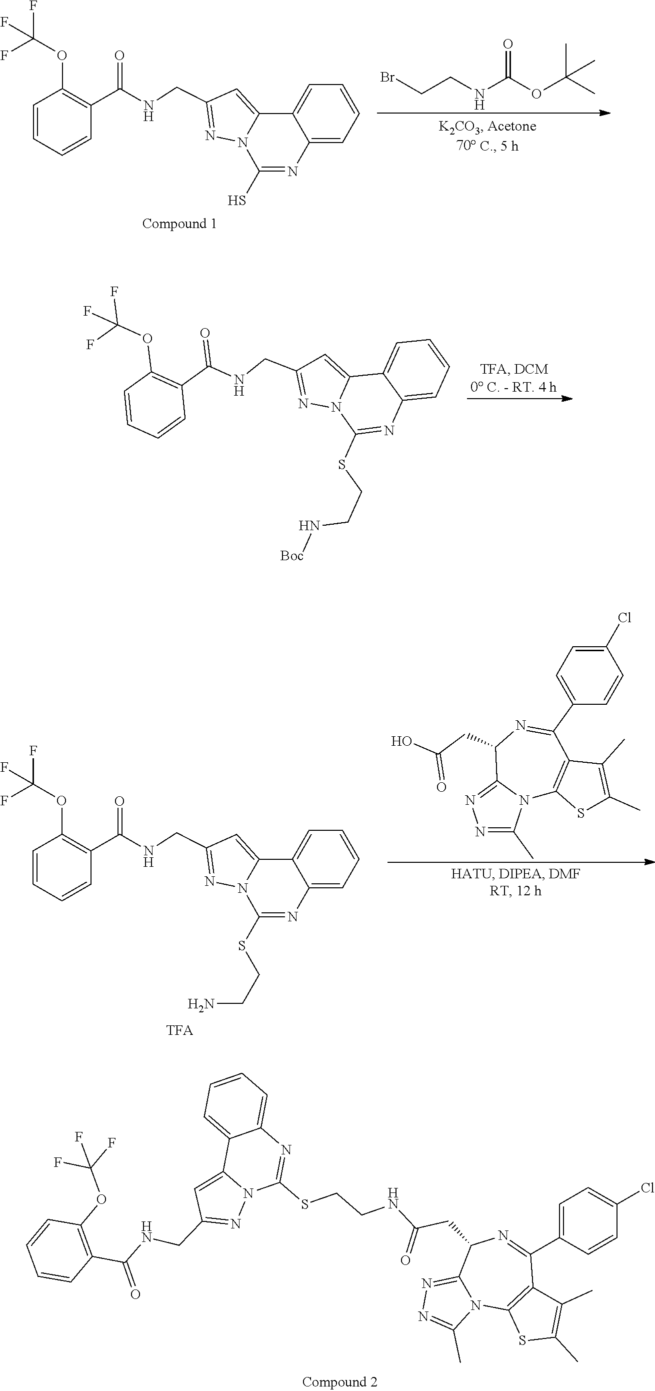 [SMILES: O=C(NCc1cc2c3ccccc3nc(S)n2n1)c1ccccc1OC(F)(F)F ; CC(C)(C)OC(=O)NCCBr ; CC(C)(C)OC(=O)NCCSc1nc2ccccc2c2cc(CNC(=O)c3ccccc3OC(F)(F)F)nn12 ; NCCSc1nc2ccccc2c2cc(CNC(=O)c3ccccc3OC(F)(F)F)nn12 ; Cc1sc2c(c1C)C(c1ccc(Cl)cc1)=N[C@@H](CC(=O)O)c1nnc(C)n1-2 ; Cc1sc2c(c1C)C(c1ccc(Cl)cc1)=N[C@@H](CC(=O)NCCSc1nc3ccccc3c3cc(CNC(=O)c4ccccc4OC(F)(F)F)nn13)c1nnc(C)n1-2]

Step-1: Synthesis of tert-butyl (2-((2-((2-(trifluoromethoxy)benzamido)methyl)pyrazolo[1,5-c]quin azolin-5-yl)thio)ethyl)carbamate.

To a stirred suspension of N-((5-mercaptopyrazolo[1,5-c]quinazolin-2-yl)methyl)-2-(trifluoro methoxy)benzamide (Compound 1, 100 mg, 0.239 mmol) in acetone (5 mL) at room temperature, was added K₂CO₃ (33 mg, 0.239 mmol) followed by tert-butyl (2-bromoethyl)carbamate (53 mg, 0.239 mmol). The resultant reaction mixture was stirred at 70° C. for 5 h. The reaction mixture was concentrated under reduced pressure and the resultant residue was subjected to column chromatography using 2-3% MeOH in dichloromethane to afford tert-butyl (2-((2-((2-(trifluoromethoxy)benzamido)methyl)pyrazolo[1,5-c]quinazolin-5-yl)thio)ethyl)carbamate off white solid (12 mg, 89.5%). ¹H NMR (400 MHZ, DMSO) δ 9.17 (t, J=6.0 Hz, 1H), 8.19 (d, J=6.7 Hz, 1H), 7.83 (d, J=8.0 Hz, 1H), 7.73-7.65 (m, 2H), 7.63-7.54 (m, 2H), 7.52-7.42 (m, 2H), 7.23 (s, 1H), 7.16 (s, 1H), 4.66 (d, J=5.9 Hz, 2H), 3.40 (dd, J=12.9, 5.0 Hz, 4H), 1.34 (s, 9H); LC-MS m/z (M+H): 561.93.

Step-2: Synthesis of N-((5-((2-aminoethyl)thio)pyrazolo[1,5-c]quinazolin-2-yl)methyl)-2-(trifluoro methoxy)benzamide 2,2,2-trifluoroacetate.

To a stirred solution of tert-butyl (2-((2-((2-(trifluoromethoxy)benzamido)methyl)pyrazolo[1,5-c]quin azolin-5-yl)thio)ethyl)carbamate (120 mg, 0.213 mmol) in DCM (5 mL) was added TFA (0.3 mL) at 0° C. The reaction mixture was warmed up and stirred at room temperature for 4 h and concentrated to afford N-((5-((2-aminoethyl)thio) pyrazolo[1,5-c]quinazolin-2-yl)methyl)-2-(trifluoromethoxy)benzamide 2,2,2-trifluoro acetate as off white solid (100 mg, 82%). ¹H NMR (400 MHZ, DMSO) δ 9.19 (t, J=6.0 Hz, 1H), 8.21 (d, J=6.6 Hz, 1H), 7.97 (d, J=16.3 Hz, 2H), 7.88 (d, J=8.2 Hz, 1H), 7.76-7.66 (m, 2H), 7.61 (dd, J=12.7, 4.7 Hz, 2H), 7.52-7.44 (m, 2H), 7.26 (s, 1H), 4.67 (d, J=6.0 Hz, 2H), 3.61 (t, J=6.6 Hz, 2H), 3.32 (dd, J=11.8, 6.0 Hz, 2H); LC-MS m/z (M+H): 462.21.

Step-3: Synthesis of(S)-N-((5-((2-(2-(4-(4-chlorophenyl)-2,3,9-trimethyl-6H-thieno[3,2-f][1,2,4]triaz olo[4,3-a][1,4]diazepin-6-yl)acetamido)ethyl)thio) pyrazolo[1,5-c]quinazolin-2-yl)methyl)-2-(trifluoro methoxy)benzamide (Compound 2):

To a stirred solution of N-((5-((2-aminoethyl)thio)pyrazolo[1,5-c]quinazolin-2-yl)methyl)-2-(trifluoromethoxy)benzamide: TFA (100 mg, 0.173 mmol) in DMF (3 mL) were added DIPEA (112 mg, 0.869 mmol), HATU (99 mg, 0.260 mmol) followed by(S)-2-(4-(4-chlorophenyl)-2,3,9-trimethyl-6H-thieno[3,2-f][1,2,4]triazolo[4,3-a][1,4]diazepin-6-yl) acetic acid (Compound 4, 83 mg, 0.208 mmol). The resultant reaction mixture was stirred at room temperature for 12 h, then diluted with cold water (20 mL) and stirred for 10 min. The resultant solid was collected by filtration, washed with water (20 mL) and dried under vacuum to afford(S)-N-((5-((2-(2-(4-(4-chlorophenyl)-2,3,9-trimethyl-6H-thieno[3,2-f][1,2,4]triazolo[4,3-a][1,4]diazepin-6-yl)acetamido)ethyl)thio)pyrazolo[1,5-c]quinazolin-2-yl)methyl)-2-(trifluoromethoxy)benzamide as white solid (50 mg, 34%). ¹H NMR (400 MHZ, DMSO) δ 9.17 (s, 1H), 8.59 (s, 1H), 8.20 (d, J=7.9 Hz, 1H), 7.82 (d, J=7.7 Hz, 1H), 7.69 (s, 2H), 7.59 (s, 2H), 7.54-7.35 (m, 6H), 7.24 (s, 1H), 4.66 (d, J=5.9 Hz, 2H), 4.51 (d, J=7.9 Hz, 1H), 3.52 (s, 4H), 3.27-3.18 (m, 2H), 2.58 (s, 3H), 2.40 (s, 4H), 1.60 (s, 3H); LC-MS m/z (M+H): 844.39.

### Synthesis of 2-((2-((2-(trifluoromethoxy)benzamido)methyl)pyrazolo[1,5-c]quinazolin-5-yl)thio)acetic acid (Compound 3)

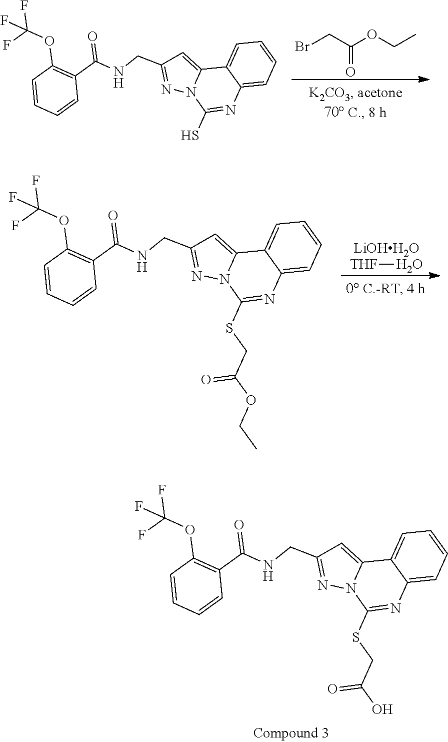

Step-1: Synthesis of ethyl 2-((2-((2-(trifluoromethoxy)benzamido)methyl) pyrazolo[1,5-c]quinazolin-5-yl)thio)acetate:

To a stirred suspension of N-((5-thioxo-5,6-dihydropyrazolo[1,5-c]quinazolin-2-yl)methyl)-2-(trifluoro methoxy)benzamide (300 mg, 0.717 mmol) in acetone (15 mL) was added K₂CO₃ (99 mg, 0.717 mmol) followed by ethyl 2-bromoacetate (87 mg, 0.717 mmol) at room temperature. The reaction mixture was stirred at 70° C. for 8 h and concentrated under reduced pressure. The residue was subjected to flash chromatography using 2-3% MeOH in DCM to afford ethyl 2-((2-((2-(trifluoromethoxy)benzamido)methyl)pyrazolo[1,5-c]quinazolin-5-yl)thio)acetate brown solid (280 mg, 77%). ¹H NMR (400 MHZ, DMSO) δ 9.17 (t, J=5.9 Hz, 1H), 8.20 (d, J=7.7 Hz, 1H), 7.74-7.66 (m, 3H), 7.64-7.57 (m, 2H), 7.52-7.42 (m, 2H), 4.68 (d, J=5.9 Hz, 2H), 4.25 (s, 2H), 4.17 (q, J=7.1 Hz, 2H), 1.21 (dd, J=9.3, 4.8 Hz, 3H); LC-MS m/z (M+H): 505.1.

Step 2: Synthesis of 2-((2-((2-(trifluoromethoxy)benzamido)methyl) pyrazolo[1,5-c]quinazolin-5-yl)thio)acetic acid (Compound 3):

To a stirred solution of ethyl 2-((2-((2-(trifluoromethoxy)benzamido)methyl) pyrazolo[1,5-c]quinazolin-5-yl)thio)acetate (280 mg, 0.55 mmol) in THF-H₂O (7 mL+3 mL) at 0° C. was added LiOH·H₂O (28 mg, 0.66 mmol) and the reaction mixture was warmed up and stirred at room temperature for 4h. It was concentrated and was diluted with H₂O (10 mL), and acidified with 1N HCl (5 mL). The solid formed was collected by filtration and dried under vacuum to afford 2-((2-((2-(trifluoromethoxy)benzamido)methyl)pyrazolo[1,5-c]quinazolin-5-yl)thio)acetic acid as white solid (200 mg, 75%). ¹H NMR (400 MHZ, DMSO) δ 9.18 (t, J=5.9 Hz, 1H), 8.19 (d, J=7.7 Hz, 1H), 7.75 (d, J=8.1 Hz, 1H), 7.69 (dd, J=10.7, 4.4 Hz, 2H), 7.64-7.55 (m, 2H), 7.50 (d, J=7.6 Hz, 1H), 7.47-7.42 (m, 1H), 7.25 (s, 1H), 4.68 (d, J=5.9 Hz, 2H), 4.19 (s, 2H); LC-MS m/z (M+H): 477.1.

### Synthesis of(S)—N-(2-aminoethyl)-2-(4-(4-chlorophenyl)-2,3,9-trimethyl-6H-thieno[3,2-f][1,2,4]triazolo[4,3-a][1,4]diazepin-6-yl)acetamideTFA salt (Compound 5)

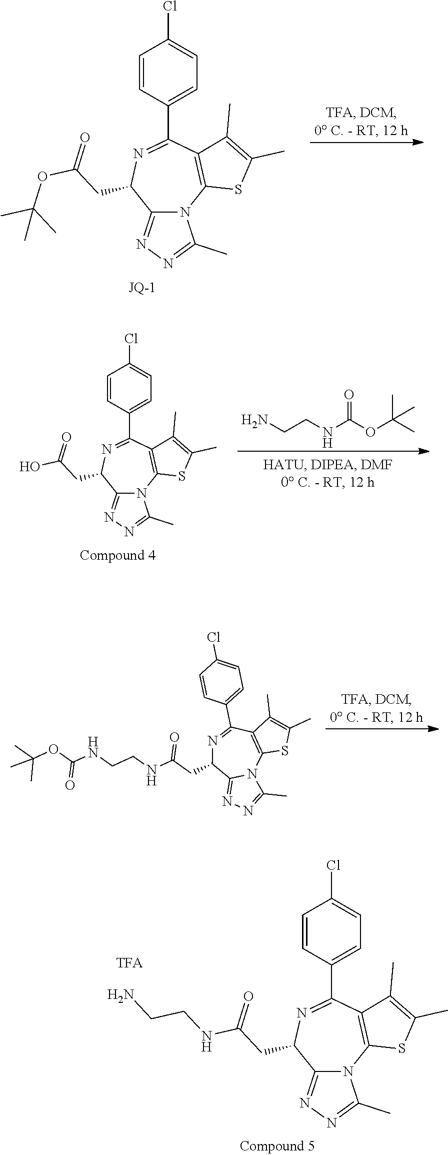

Step-1: Synthesis of(S)-2-(4-(4-chlorophenyl)-2,3,9-trimethyl-6H-thieno[3,2-f][1,2,4]triazolo[4,3-a][1,4]diazepin-6-yl)acetic acid (Compound 4):

To a stirred solution of tert-butyl(S)-2-(4-(4-chlorophenyl)-2,3,9-trimethyl-6H-thieno[3,2-f][1,2,4]triazolo[4,3-a][1,4]diazepin-6-yl)acetate (500 mg, 1.094 mmol) in DCM (20 mL) at 0° C. was added TFA (1 mL). The reaction mixture was warmed up and stirred at room temperature for 12 h. The resultant reaction mixture was concentrated and co-distilled twice with DCM (10 mL each) to afford(S)-2-(4-(4-chlorophenyl)-2,3,9-trimethyl-6H-thieno[3,2-f][1,2,4]triazolo[4,3-a][1,4]diazepin-6-yl)acetic acid: TFA salt as a pale yellow gummy solid (400 mg, 91%). LC-MS: m/z=401.20

Step-2: Synthesis of tert-butyl(S)-(2-(2-(4-(4-chlorophenyl)-2,3,9-trimethyl-6H-thieno[3,2-f][1,2,4]triazolo[4,3-a][1,4]diazepin-6-yl)acetamido)ethyl)carbamate:

To a stirred solution of(S)-2-(4-(4-chlorophenyl)-2,3,9-trimethyl-6H-thieno[3,2-f][1,2,4]triazolo[4,3-a][1,4]diazepin-6-yl)acetic acid: TFA (250 mg, 0.623 mmol) and tert-butyl (2-aminoethyl)carbamate (150 mg, 0.935 mmol) in DMF (5 mL) at 0° C. were added HATU (355 mg, 0.935 mmol) followed by DIPEA (0.16 mL, 0.935 mmol). The resultant reaction mixture was stirred at room temperature for 12 h, then was quenched with ice cold water (20 mL) and extracted with EtOAc (3×20 mL). The combined organic layer was dried over Na₂SO₄ and concentrated under reduced pressure. The residue was purified by flash chromatography using 3% MeOH in DCM to afford tert-butyl(S)-(2-(2-(4-(4-chlorophenyl)-2,3,9-trimethyl-6H-thieno[3,2-f][1,2,4]triazolo[4,3-a][1,4]diazepin-6-yl)acetamido)ethyl) carbamate as a pale-yellow gummy solid (110 mg, 32.4%). LC-MS: m/z=543.1.

Step-3: Synthesis of(S)—N-(2-aminoethyl)-2-(4-(4-chlorophenyl)-2,3,9-trimethyl-6H-thieno[3,2-f][1,2,4]triazolo[4,3-a][1,4]diazepin-6-yl) acetamide: TFA salt (Compound 5):

To a stirred solution of tert-butyl(S)-(2-(2-(4-(4-chlorophenyl)-2,3,9-trimethyl-6H-thieno[3,2-f][1,2,4]triazolo[4,3-a][1,4]diazepin-6-yl)acetamido)ethyl)carbamate (100 mg, 0.181 mmol) in DCM (5 mL) at 0° C. was added TFA (0.2 mL). The reaction mixture was warmed up and stirred at room temperature for 12h. It was then concentrated and residue was co-distilled twice (to remove residual TEA) with DCM (10 mL) to afford(S)—N-(2-aminoethyl)-2-(4-(4-chlorophenyl)-2,3,9-trimethyl-6H-thieno[3,2-f][1,2,4]triazolo[4,3-a][1,4]diazepin-6-yl) acetamide as a pale yellow gummy solid (78 mg, 97.5%). LC-MS: m/z=443.2.

### Synthesis of(S)-N-((5-((2-((2-(2-(4-(4-chlorophenyl)-2,3,9-trimethyl-6H-thieno[3,2-f][1,2,4]triazolo[4,3-a][1,4]diazepin-6-yl)acetamido)ethyl)amino)-2-oxoethyl)thio)pyrazolo[1,5-c]quinazolin-2-yl)methyl)-2-(trifluoromethoxy)benzamide (Compound 6)

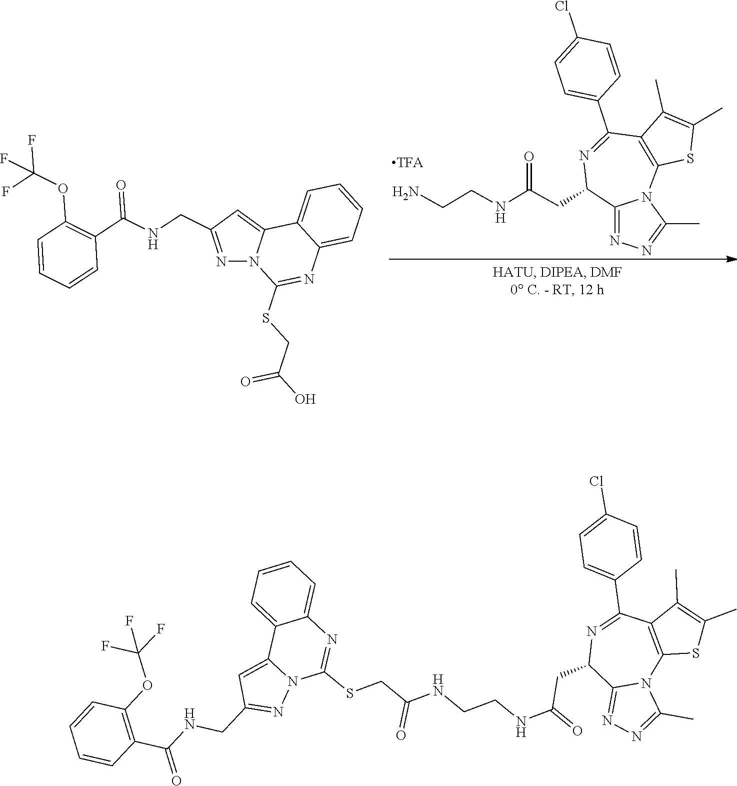 [SMILES: O=C(O)CSc1nc2ccccc2c2cc(CNC(=O)c3ccccc3OC(F)(F)F)nn12 ; Cc1sc2c(c1C)C(c1ccc(Cl)cc1)=N[C@@H](CC(=O)NCCN)c1nnc(C)n1-2 ; Cc1sc2c(c1C)C(c1ccc(Cl)cc1)=N[C@@H](CC(=O)NCCNC(=O)CSc1nc3ccccc3c3cc(CNC(=O)c4ccccc4OC(F)(F)F)nn13)c1nnc(C)n1-2]

To a stirred solution of 2-((2-((2-(trifluoromethoxy)benzamido)methyl) pyrazolo[1,5-c]quinazolin-5-yl)thio)acetic acid (100 mg, 0.209 mmol) in DMF (3 mL) were added DIPEA (40.6 mg, 0.314 mmol) and HATU (119.6 mg, 0.314 mmol) followed by(S)-N-(2-aminoethyl)-2-(4-(4-chlorophenyl)-2,3,9-trimethyl-6H-thieno[3,2-f][1,2,4]triazolo[4,3-a][1,4]diazepin-6-yl) acetamide: TFA salt (139 mg, 0.314 mmol). The reaction mixture stirred at room temperature for 12 h, then diluted water (20 mL) and extracted with EtOAc (2×20 mL).

The combined organic layer was dried over anhydrous Na₂SO₄ and concentrated under reduced pressure. The residue was purified by prep-HPLC to afford(S)-N-((5-((2-((2-(2-(4-(4-chlorophenyl)-2,3,9-trimethyl-6H-thieno[3,2-f][1,2,4]triazolo[4,3-a][1,4]diazepin-6-yl) acetamido)ethyl)amino)-2-oxoethyl)thio)pyrazolo[1,5-c]quinazolin-2-yl)methyl)-2-(trifluoromethoxy)benzamide as an off-white solid (35 mg, 18.5%). ¹H NMR (400 MHZ, DMSO-d₆) δ 9.17 (t, J=5.6 Hz, 1H), 8.34 (bs, 1H), 8.25 (bs, 1H), 8.16 (d, J=7.6 Hz, 1H), 7.78 (d, J=8.0 Hz, 1H), 7.69-7.54 (m, 4H), 7.50-7.38 (m, 6H), 7.22 (s, 1H), 4.67 (d, J=6.0 Hz, 2H), 4.44 (t, J=7.2 Hz, 1H), 4.11 (s, 2H), 3.20-3.16 (m, 4H), 2.57 (s, 3H), 2.39 (s, 3H), 1.60 (s, 3H); LC-MS: m/z=901.53.

### Synthesis of(S)-N-((5-((2-((2-(2-(2-(4-(4-chlorophenyl)-2,3,9-trimethyl-6H-thieno[3,2-f][1,2,4]triazolo[4,3-a][1,4]diazepin-6-yl)acetamido) ethoxy)ethyl)amino)-2-oxoethyl)thio)pyrazolo[1,5-c]quinazolin-2-yl)methyl)-2-(trifluoromethoxy)benzamide (Compound 7)

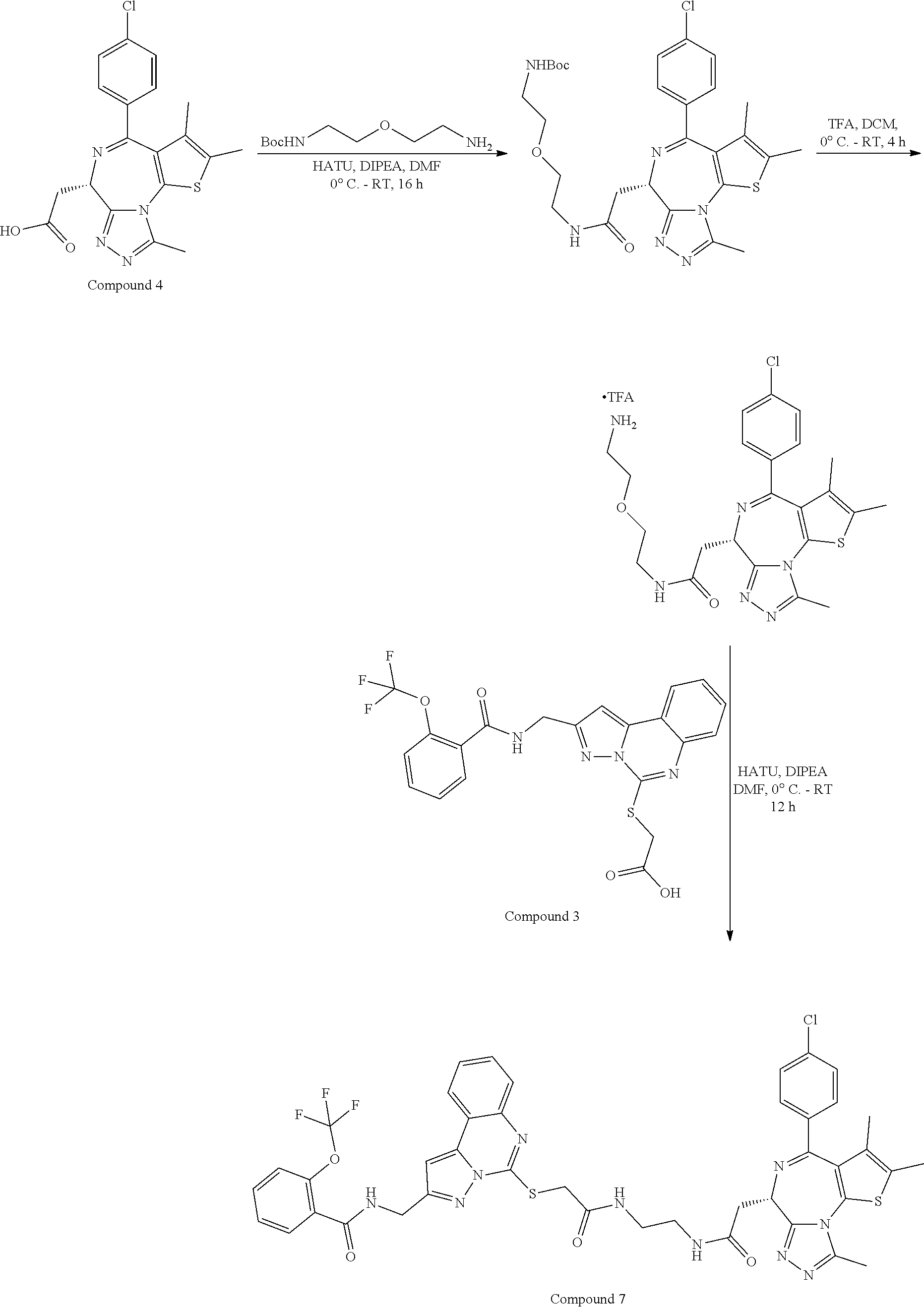 [SMILES: Cc1sc2c(c1C)C(c1ccc(Cl)cc1)=N[C@@H](CC(=O)O)c1nnc(C)n1-2 ; CC(C)(C)OC(=O)NCCOCCN ; Cc1sc2c(c1C)C(c1ccc(Cl)cc1)=N[C@@H](CC(=O)NCCOCCNC(=O)OC(C)(C)C)c1nnc(C)n1-2 ; Cc1sc2c(c1C)C(c1ccc(Cl)cc1)=N[C@@H](CC(=O)NCCOCCN)c1nnc(C)n1-2 ; O=C(O)CSc1nc2ccccc2c2cc(CNC(=O)c3ccccc3OC(F)(F)F)nn12 ; Cc1sc2c(c1C)C(c1ccc(Cl)cc1)=N[C@@H](CC(=O)NCCNC(=O)CSc1nc3ccccc3c3cc(CNC(=O)c4ccccc4OC(F)(F)F)nn13)c1nnc(C)n1-2]

Step 1: Synthesis of tert-butyl(S)-(2-(2-(2-(4-(4-chlorophenyl)-2,3,9-trimethyl-6H-thieno[3,2-f][1,2,4]triazolo[4,3-a][1,4]diazepin-6-yl)acetamido) ethoxy)ethyl) carbamate:

A solution of(S)-2-(4-(4-chlorophenyl)-2,3,9-trimethyl-6H-thieno[3,2-f][1,2,4]triazolo[4,3-a][1,4]diazepin-6-yl)acetic acid (400 mg, 1.0 mmol), DIPEA (0.52 mL, 3.0 mmol), HATU (570 mg, 1.5 mmol) and tert-butyl (2-(2-aminoethoxy)ethyl)carbamate (245 mg, 1.2 mmol) was stirred at room temperature for 16 h. The mixture was diluted with ice-cold water (50 mL) and extracted with EtOAc (2×50 mL). The combined organic layer was washed with ice water (2×50 mL) and brine (50 mL), dried over Na₂SO₄ and concentrated under vacuum. The crude compound was purified by 0-5% MeOH in dichloromethane to afford tert-butyl(S)-(2-(2-(2-(4-(4-chlorophenyl)-2,3,9-trimethyl-6H-thieno[3,2-f][1,2,4]triazolo[4,3-a][1,4]diazepin-6-yl)acetamido) ethoxy)ethyl)carbamate as an off white solid (290 mg, 49%). LC-MS (ESI): m/z 587.1 (M+H)

Step 2: Synthesis of(S)—N-(2-(2-aminoethoxy)ethyl)-2-(4-(4-chlorophenyl)-2,3,9-trimethyl-6H-thieno[3,2-f][1,2,4]triazolo[4,3-a][1,4]diazepin-6-yl) acetamide: TFA.

To a stirred solution of tert-butyl(S)-(2-(2-(2-(4-(4-chlorophenyl)-2,3,9-trimethyl-6H-thieno[3,2-f][1,2,4]triazolo[4,3-a][1,4]diazepin-6-yl)acetamido) ethoxy)ethyl) carbamate (290 mg, 0.49 mmol) in DCM (3 mL) was added TFA (0.5 mL) at 0° C. and the mixture was stirred at room temperature for 4 h. After completion of reaction, the reaction mixture was concentrated under vacuum. The crude compound was purified by preparative HPLC (water; ACN) to afford(S)—N-(2-(2-aminoethoxy)ethyl)-2-(4-(4-chlorophenyl)-2,3,9-trimethyl-6H-thieno[3,2-f][1,2,4]triazolo[4,3-a][1,4]diazepin-6-yl) acetamide. TFA as pale yellow solid (200 mg, 83%). ¹H NMR (400 MHZ, DMSO) δ 8.25 (d, J=5.3 Hz, 1H), 7.79 (s, 3H), 7.56-7.47 (m, 2H), 7.42 (d, J=8.4 Hz, 2H), 4.52 (t, J=7.1 Hz, 1H), 3.61 (d, J=5.2 Hz, 2H), 3.49 (t, J=5.5 Hz, 2H), 3.38-3.24 (m, 4H), 3.08-2.97 (m, 2H), 2.61 (d, J=5.3 Hz, 3H), 2.42 (d, J=4.5 Hz, 3H), 1.62 (s, 3H); LC-MS (ESI): m/z 487.4 (M+H)⁺.

### Step-3: Synthesis of(S)-N-((5-((2-((2-(2-(2-(4-(4-chlorophenyl)-2,3,9-trimethyl-6H-thieno[3,2-f][1,2,4]triazolo[4,3-a][1,4]diazepin-6-yl)acetamido) ethoxy)ethyl)amino)-2-oxoethyl)thio)pyrazolo[1,5-c]quinazolin-2-yl)methyl) ˜2-(trifluoromethoxy)benzamide

To a stirred solution of 2-((2-((2-(trifluoromethoxy)benzamido)methyl) pyrazolo[1,5-c]quinazolin-5-yl)thio)acetic acid (100 mg, 0.209 mmol) in DMF (1.5 mL) were added DIPEA (40.6 mg, 0.314 mmol) and HATU (119.6 mg, 0.314 mmol) followed by(S)-N-(2-(2-aminoethoxy)ethyl)-2-(4-(4-chlorophenyl)-2,3,9-trimethyl-6H-thieno[3,2-f][1,2,4]triazolo[4,3-a][1,4]diazepin-6-yl) acetamide: TFA salt (153 mg, 0.314 mmol). The reaction mixture stirred at room temperature for 12 h, then diluted water (30 mL) and extracted with EtOAc (2×20 mL). The combined organic layer was dried over anhydrous Na₂SO₄ and concentrated under reduced pressure. The residue was purified by prep-HPLC to afford(S)-N-((5-((2-((2-(2-(2-(4-(4-chlorophenyl)-2,3,9-trimethyl-6H-thieno[3,2-f][1,2,4]triazolo[4,3-a][1,4]diazepin-6-yl)acetamido) ethoxy)ethyl)amino)-2-oxoethyl)thio)pyrazolo[1,5-c]quinazolin-2-yl)methyl)-2-(trifluoromethoxy)benzamide as an off-white solid (39 mg, 19.6%). ¹H NMR (400 MHZ, DMSO-d₆) δ 9.18 (t, J=5.6 Hz, 1H), 8.41-8.38 (m, 2H), 8.17 (d, J=7.2 Hz, 1H), 7.67 (t, J=7.2 Hz, 2H), 7.62-7.60 (m, 3H), 7.58-7.54 (m, 4H), 7.38 (d, J=8.4 Hz, 2H), 7.23 (s, 1H), 4.67 (d, J=6.0 Hz, 2H), 4.57 (t, J=8 Hz, 1H), 4.16-4.07 (m, 2H), 3.49-3.44 (m, 4H), 3.15-3.31 (m, 6H), 2.45 (s, 3H), 2.26 (s, 3H), 1.53 (s, 3H). LC-MS: m/z=945.41 (M+H)+

### Synthesis of(S)-N-((5-((14-(4-(4-chlorophenyl)-2,3,9-trimethyl-6H-thieno[3,2 f][1,2,4]triazolo[4,3-a][1,4]diazepin-6-yl)-2,13-dioxo-6,9-dioxa-3,12-diazatetradecyl)thio)pyrazolo[1,5-c]quinazolin-2-yl)methyl)-2-(trifluoromethoxy)benzamide (Compound 9)

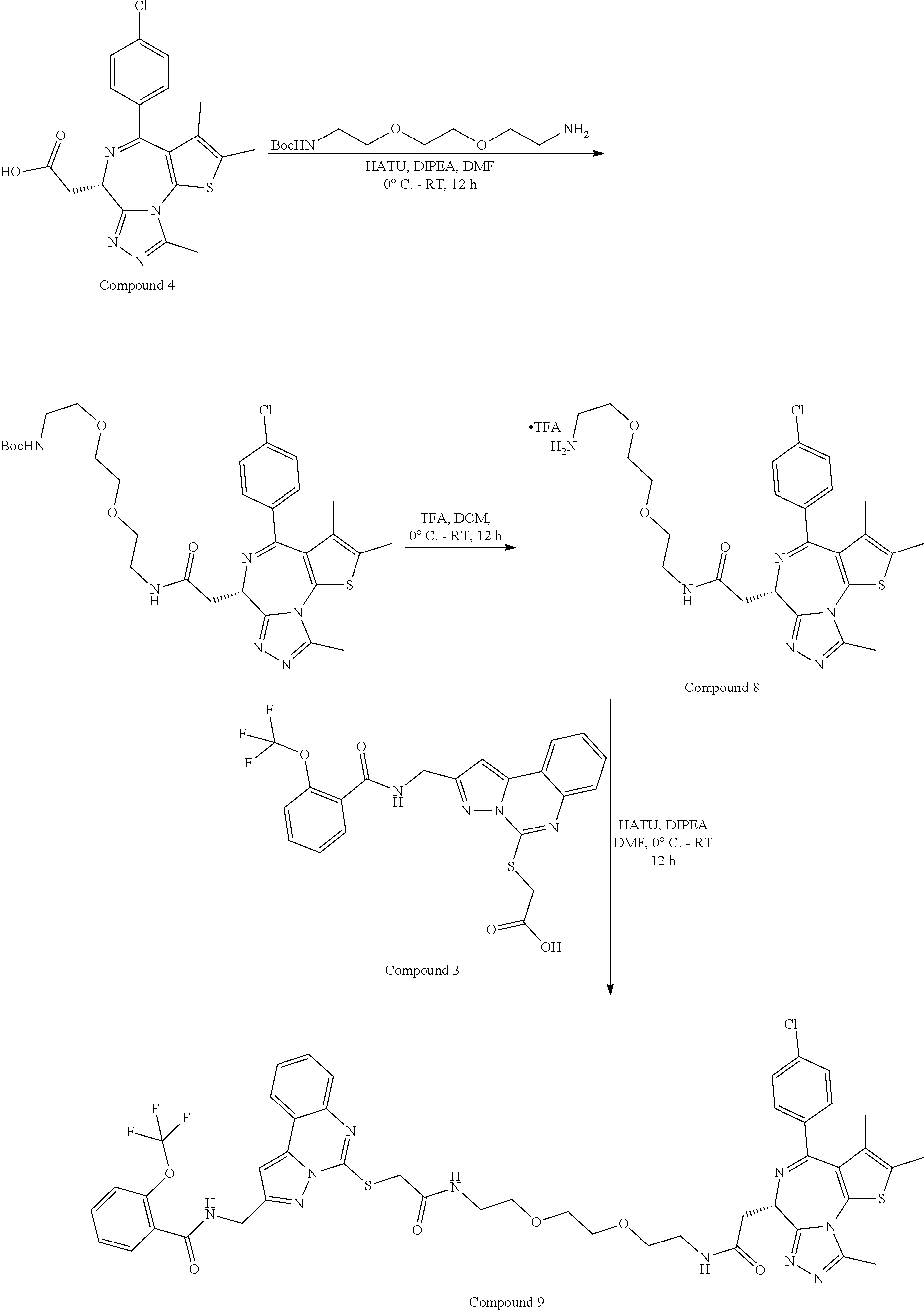 [SMILES: Cc1sc2c(c1C)C(c1ccc(Cl)cc1)=N[C@@H](CC(=O)O)c1nnc(C)n1-2 ; CC(C)(C)OC(=O)NCCOCCOCCN ; Cc1sc2c(c1C)C(c1ccc(Cl)cc1)=N[C@@H](CC(=O)NCCOCCOCCNC(=O)OC(C)(C)C)c1nnc(C)n1-2 ; Cc1sc2c(c1C)C(c1ccc(Cl)cc1)=N[C@@H](CC(=O)NCCOCCOCCN)c1nnc(C)n1-2 ; O=C(O)CSc1nc2ccccc2c2cc(CNC(=O)c3ccccc3OC(F)(F)F)nn12 ; Cc1sc2c(c1C)C(c1ccc(Cl)cc1)=N[C@@H](CC(=O)NCCOCCOCCNC(=O)CSc1nc3ccccc3c3cc(CNC(=O)c4ccccc4OC(F)(F)F)nn13)c1nnc(C)n1-2]

Step-1: Synthesis of tert-butyl(S)-(2-(2-(2-(2-(4-(4-chlorophenyl)-2,3,9-trimethyl-6H-thieno[3,2-f][1,2,4]triazolo[4,3-a][1,4]diazepin-6-yl)acetamido) ethoxy) ethoxy)ethyl)carbamate:

To a stirred solution of(S)-2-(4-(4-chlorophenyl)-2,3,9-trimethyl-6H-thieno[3,2-f][1,2,4]triazolo[4,3-a][1,4]diazepin-6-yl)acetic acid (860 mg, 2.145 mmol) and tert-butyl (2-(2-(2-aminoethoxy) ethoxy)ethyl)carbamate (799 mg, 3.217 mmol) in DMF (10 mL) at 0° C. were added HATU (1.2 g, 3.217 mmol) followed by DIPEA (0.53 mL, 3.217 mmol). The reaction mixture was stirred at room temperature for 12 h. After completion of reaction, the mixture was quenched with ice cold water (30 mL) and extracted with EtOAc (3×30 mL). The combined organic layer was dried over Na₂SO₄ and concentrated under reduced pressure. The residue was purified by flash chromatography using 3% MeOH in DCM to afford tert-butyl(S)-(2-(2-(2-(2-(4-(4-chlorophenyl)-2,3,9-trimethyl-6H-thieno[3,2-f][1,2,4]triazolo[4,3-a][1,4]diazepin-6-yl)acetamido) ethoxy) ethoxy)ethyl)carbamate as a pale-yellow gummy solid (590 mg, 43.7%). LC-MS: m/z=631.26 (M+H)⁺.

Step-2: Synthesis of(S)—N-(2-(2-(2-aminoethoxy) ethoxy)ethyl)-2-(4-(4-chlorophenyl)-2,3,9-trimethyl-6H-thieno[3,2-f][1,2,4]triazolo[4,3-a][1,4]diazepin-6-yl) acetamide: TFA salt,

To a stirred solution of tert-butyl(S)-(2-(2-(2-(2-(4-(4-chlorophenyl)-2,3,9-trimethyl-6H-thieno[3,2-f][1,2,4]triazolo[4,3-a][1,4]diazepin-6-yl)acetamido) ethoxy) ethoxy)ethyl)carbamate (590 mg, 0.934 mmol) in DCM (10 mL) at 0° C. was added TFA (2 mL). The reaction mixture was warmed up and stirred at room temperature for 12 h. The reaction solvent was evaporated and co-distilled twice with DCM (10 mL) to afford(S)—N-(2-(2-(2-aminoethoxy) ethoxy)ethyl)-2-(4-(4-chlorophenyl)-2,3,9-trimethyl-6H-thieno[3,2-f][1,2,4]triazolo[4,3-a][1,4]diazepin-6-yl) acetamideTFA salt as a pale-yellow gummy solid (423 mg, 85%). JH NMR (400 MHZ, DMSO) δ 8.30 (t, J=5.6 Hz, 1H), 7.81 (bs, 3H), 7.56-7.48 (m, 2H), 7.47-7.38 (m, 2H), 4.55-4.50 (m, 1H), 3.61 (d, J=5.6, 8.7 Hz, 6H), 3.47 (t, J=5.6 Hz, 2H), 3.31-3.25 (m, 4H), 3.02-2.98 (m, 2H), 2.31 (s, 3H), 2.42 (s, 3H), 1.64 (s, 3H); LC-MS: m/z=531.3 (530+H)⁺.

Step-3: Synthesis of(S)-N-((5-((14-(4-(4-chlorophenyl)-2,3,9-trimethyl-6H-thieno[3,2 f][1,2,4]triazolo[4,3-a][1,4]diazepin-6-yl)-2,13-dioxo-6,9-dioxa-3,12-diazatetradecyl)thio)pyrazolo[1,5-c]quinazolin-2-yl)methyl)-2-(trifluoromethoxy)benzamide:

To a stirred solution of 2-((2-((2-(trifluoromethoxy)benzamido)methyl) pyrazolo[1,5-c]quinazolin-5-yl)thio)acetic acid (100 mg, 0.210 mmol) in DMF (1.5 mL) were added DIPEA (40.6 mg, 0.315 mmol) and HATU (119.7 mg, 0.315 mmol) followed by(S)-N-(2-(2-(2-aminoethoxy) ethoxy)ethyl)-2-(4-(4-chlorophenyl)-2,3,9-trimethyl-6H-thieno[3,2-f][1,2,4]triazole[4,3-a][1,4]diazepin-6-yl) acetamide: TFA salt (167.2 mg, 0.315 mmol). The resultant reaction mixture stirred at room temperature for 12 h, then diluted water (30 mL) and extracted with EtOAc (2×20 mL). The combined organic layer was dried over anhydrous Na₂SO₄ and concentrated under reduced pressure. The residue was purified by prep-HPLC to afford(S)-N-((5-((14-(4-(4-chlorophenyl)-2,3,9-trimethyl-6H-thieno[3,2-f][1,2,4]triazolo[4,3-a][1,4]diazepin-6-yl)-2,13-dioxo-6,9-dioxa-3,12-diazatetradecyl)thio) pyrazolo[1,5-c]quinazolin-2-yl)methyl)-2-(trifluoromethoxy)benzamide as an off white solid (88.9 mg, 42.78%). ¹H NMR (400 MHZ, DMSO-d₆) δ 9.17 (t, J=6.0 Hz, 1H), 8.37 (t, J=5.6 Hz, 1H), 8.27 (t, J=5.6 Hz, 1H), 8.19 (d, J=8.0 Hz, 1H), 7.77 (d, J=8.0 Hz, 1H), 7.69-7.65 (m, 2H), 7.62-7.55 (m, 2H), 7.50-7.38 (m, 6H), 7.24 (s, 1H), 4.68 (d, J=5.6 Hz, 2H), 4.50 (t, J=7.2 Hz, 1H), 4.10 (s, 2H), 3.48 (s, 4H), 3.42-3.40 (m, 4H), 3.29-3.21 (m, 6H), 2.55 (s, 3H), 2.36 (s, 3H), 1.58 (s, 3H); LC-MS: m/z=989.45 (M+H)⁺.

### Synthesis of (R)-N-((5-(2-((14-(4-(4-chlorophenyl)-2,3,9-trimethyl-6H-thieno[3,2-f][1,2,4]triazolo[4,3-a][1,4]diazepin-6-yl)-2,13-dioxo-6,9-dioxa-3,12-diazatetradecyl)oxy)phenyl)-1H-pyrazol-3-yl)methyl)-2-(trifluoromethoxy)benzamide (Compound 10)

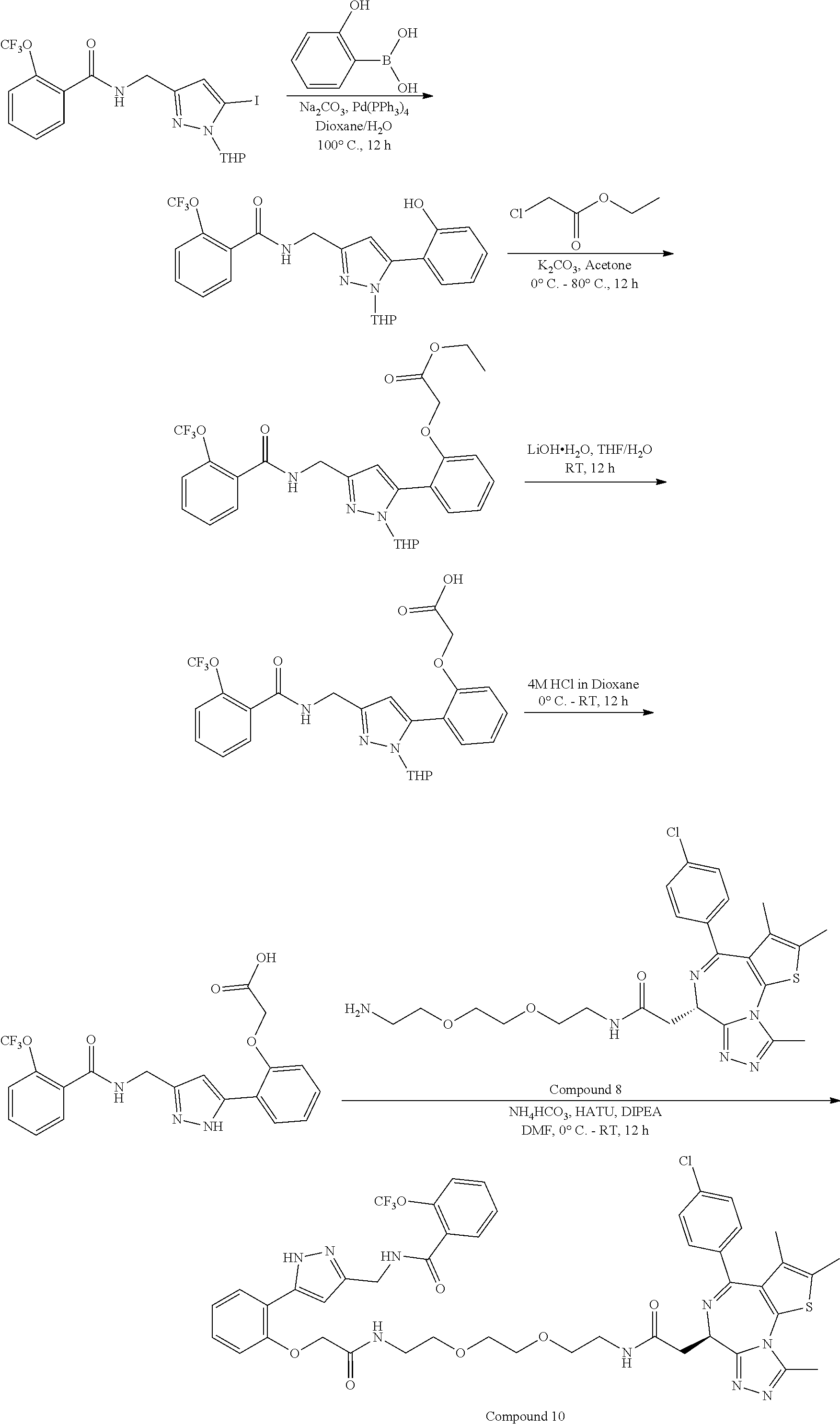

Step-1: Synthesis of N-((5-(2-hydroxyphenyl)-1-(tetrahydro-2H-pyran-2-yl)-1H-pyrazol-3-yl)methyl)-2-(trifluoromethoxy)benzamide:

To a stirred solution of N-((5-iodo-1-(tetrahydro-2H-pyran-2-yl)-1H-pyrazol-3-yl)methyl)-2-(trifluoromethoxy)benzamide (250 mg, 0.505 mmol), (2-hydroxyphenyl) boronic acid (83.5 mg, 0.606 mmol) in 1,4-dioxane (16 mL) and water (4 mL) was added Na₂CO₃ (133.7 mg, 1.262 mmol) and the resultant mixture was degassed with argon gas for 5 min, followed by the addition of Pd(PPh₃)₄ (58.3 mg, 0.05 mmol). The resultant reaction mixture was stirred at 100° C. for 12 h and it was concentrated. The residue obtained was diluted with water (30 mL) and extracted with EtOAc (3×20 mL). The combined organic layer was dried over Na₂SO₄ and concentrated under reduced pressure. The crude product obtained was purified by flash chromatography using 30% EtOAc in hexane to afford N-((5-(2-hydroxy phenyl)-1-(tetrahydro-2H-pyran-2-yl)-1H-pyrazol-3-yl)methyl)-2-(trifluoro methoxy)benzamide as an off-white solid (160 mg, 68.6%). LC-MS: m/z=460.43 (M−H)⁺.

Step-2: Synthesis of ethyl 2-(2-(1-(tetrahydro-2H-pyran-2-yl)-3-((2-(trifluoromethoxy)benzamido)methyl)-1H-pyrazol-5-yl) phenoxy)acetate:

To a stirred solution of N-((5-(2-hydroxyphenyl)-1-(tetrahydro-2H-pyran-2-yl)-1H-pyrazol-3-yl)methyl)-2-(trifluoromethoxy)benzamide (160 mg, 0.346 mmol) in acetone (7 mL) was added K₂CO₃ (47.8 mg, 0.346 mmol) followed by ethyl 2-chloroacetate (42.4 mg, 0.346 mmol). The reaction mixture was stirred at 80° C. for 12 h and concentrated. The residue was then diluted with water (20 mL) and extracted with EtOAc (2×20 mL). The combined organic layer was dried over Na₂SO₄ and concentrated under reduced pressure. The crude product was purified by flash chromatography using 30% EtOAc in hexane to afford ethyl 2-(2-(1-(tetrahydro-2H-pyran-2-yl)-3-((2-(trifluoromethoxy)benzamido)methyl)-1H-pyrazol-5-yl) phenoxy)acetate a yellow gummy solid (105 mg, 55.3%). LC-MS: m/z=546.35 (M−H)⁺.

Step-3: Synthesis of 2-(2-(1-(tetrahydro-2H-pyran-2-yl)-3-((2-(trifluoromethoxy)benzamido)methyl)-1H-pyrazol-S-yl) phenoxy)acetic acid.

To a stirred solution of ethyl 2-(2-(1-(tetrahydro-2H-pyran-2-yl)-3-((2-(trifluoromethoxy)benzamido)methyl)-1H-pyrazol-5-yl) phenoxy)acetate (105 mg, 0.191 mmol) in THF (2.5 mL) and water (2.5 mL) was added LiOH·H₂O (30 mg, 0.767 mmol). The reaction mixture was stirred at room temperature for 12 h and it was extracted with EtOAc (20 mL). The aqueous layer was acidified with 1N HCl and extracted with EtOAc (3×20 mL). The combined organic layer was dried over Na₂SO₄ and concentrated under reduced pressure. The crude compound was triturated with diethyl ether (10 mL) to afford 2-(2-(1-(tetrahydro-2H-pyran-2-yl)-3-((2-(trifluoromethoxy)benzamido)methyl)-1H-pyrazol-5-yl) phenoxy)acetic acid as an off white solid (95 mg, 95.3%). LC-MS: m/z=518.4 (M−H)⁺.

Step-4: Synthesis of 2-(2-(3-((2-(trifluoromethoxy)benzamido)methyl)-1H-pyrazol-5-yl) phenoxy)acetic acid:

To a stirred solution of 2-(2-(1-(tetrahydro-2H-pyran-2-yl)-3-((2-(trifluoromethoxy)benzamido)methyl)-1H-pyrazol-5-yl) phenoxy)acetic acid (140 mg, 0.282 mmol) in DCM (5 mL) at 0° C. was added 4M HCl in 1,4-Dioxane (5 mL). The reaction mixture was stirred at room temperature for 12 h. After completion of reaction, the solvent was distilled off and the residue was neutralized with saturated NaHCO₃ solution and stirred for 10 min. The obtained solid was collected by filtration and dried under vacuum to afford 2-(2-(3-((2-(trifluoromethoxy)benzamido)methyl)-1H-pyrazol-5-yl) phenoxy)acetic acid (95 mg, 73%) as an off-white solid (95 mg, 73%). ¹H NMR (400 MHz, DMSO-d₆) $15.26 (s, 1H), 8.87 (t, J=5.6 Hz, 1H), 7.68-7.54 (m, 3H), 7.46 (q, J=15.2 Hz, 2H), 7.25 (t, J=7.2 Hz, 1H), 7.09 (d, J=8.4 Hz, 1H), 7.00 (t, J=7.6 Hz, 1H), 6.55 (s, 1H), 4.44 (d, J=5.6 Hz, 2H), 4.34 (s, 2H); LC-MS: m/z=436.2 (435+H)⁺.

Step-S: Synthesis of (R)-N-((5-(2-((14-(4-(4-chlorophenyl)-2,3,9-trimethyl-6H-thieno[3,2-f][1,2,4]triazolo[4,3-a][1,4]diazepin-6-yl)-2,13-dioxo-6,9-dioxa-3,12-diazatetradecyl)oxy)phenyl)-1H-pyrazol-3-yl)methyl)-2-(trifluoromethoxy)benzamide:

To a stirred solution of 2-(2-(3-((2-(trifluoromethoxy)benzamido)methyl)-1H-pyrazol-5-yl) phenoxy)acetic acid (70 mg, 0.160 mmol) and(S)—N-(2-(2-(2-aminoethoxy) ethoxy)ethyl)-2-(4-(4-chlorophenyl)-2,3,9-trimethyl-6H-thieno[3,2-f][1,2,4]triazolo[4,3-a][1,4]diazepin-6-yl) acetamide (155.5 mg, 0.241 mmol) in DMF (3 mL) at 0° C. were added HATU (91.6 mg, 0.241 mmol) followed by DIPEA (62 mg, 0.482 mmol). The reaction mixture was stirred at room temperature for 12 h, then quenched with ice-cold water (30 mL) and extracted with EtOAc (3×20 mL). The combined organic layer was dried over Na₂SO₄, and concentrated under reduced pressure. The residue was purified by prep-HPLC to afford (R)-N-((5-(2-((14-(4-(4-chlorophenyl)-2,3,9-trimethyl-6H-thieno[3,2-f][1,2,4]triazolo[4,3-a][1,4]diazepin-6-yl)-2,13-dioxo-6,9-dioxa-3,12-diazatetradecyl)oxy)phenyl)-1H-pyrazol-3-yl)methyl)-2-(trifluoromethoxy)benzamide (25 mg, 16%) as an off-white solid. ¹H NMR (400 MHZ, DMSO-d₆) § 13.0 (s, 1H), 8.97-8.76 (m, 1H), 8.34-8.20 (m, 2H), 7.71-7.55 (m, 3H), 7.47-7.39 (m, 6H), 7.28 (t, J=7.6 Hz, 1H), 7.06-6.97 (m, 2H), 6.64 (s, 1H), 4.68 (s, 1H), 4.55-4.31 (m, 4H), 3.50 (s, 4H), 3.45-3.41 (m, 4H), 3.24-3.22 (m, 4H), 2.57 (s, 3H), 2.38 (s, 3H), 1.59 (s, 3H); LC-MS: m/z=948.5 (M+H)⁺.

### Synthesis of(S)-N-((1-(1-(4-(4-chlorophenyl)-2,3,9-trimethyl-6H-thieno[3,2-f][1,2,4]triazolo[4,3-a][1,4]diazepin-6-yl)-2,13-dioxo-6,9-dioxa-3,12-diazapentadecan-15-yl)-S-(thiophen-2-yl)-1H-pyrazol-3-yl)methyl)-2-(trifluoromethoxy)benzamide (Compound 11)

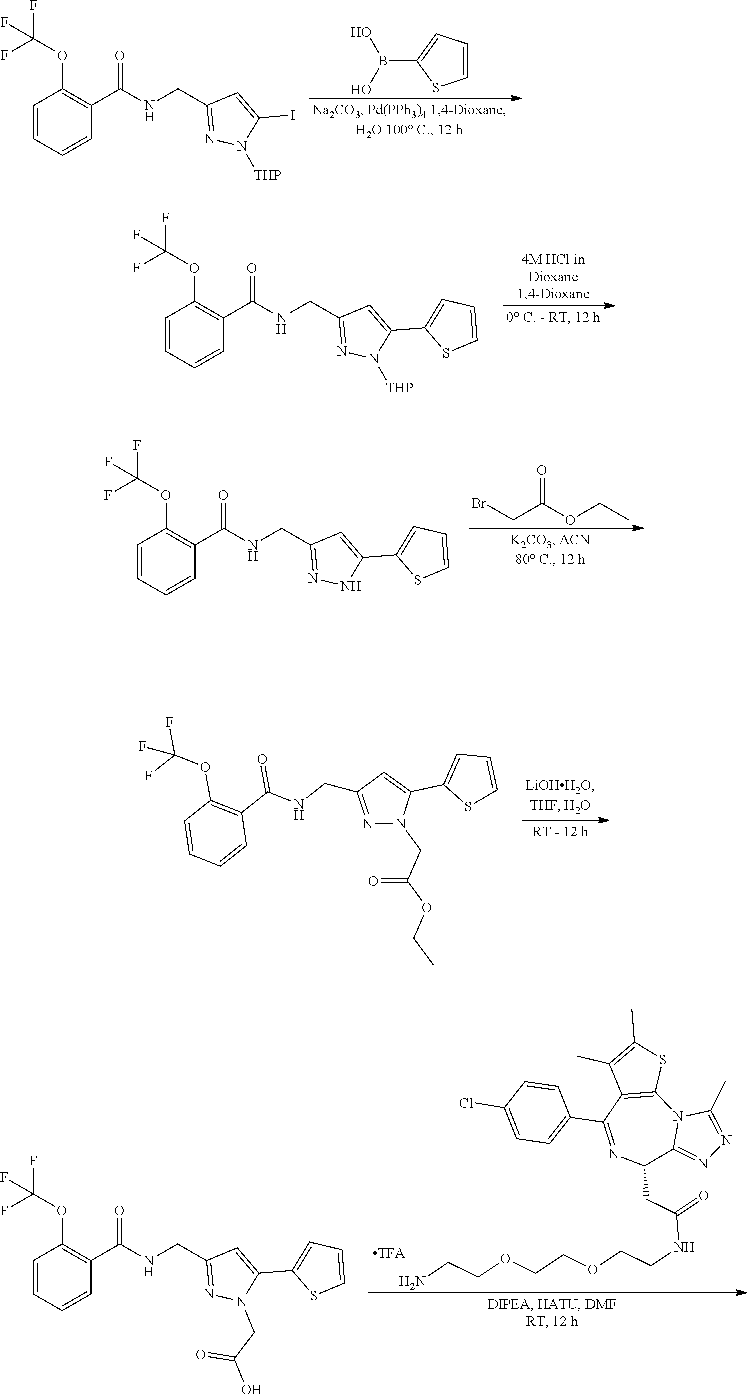

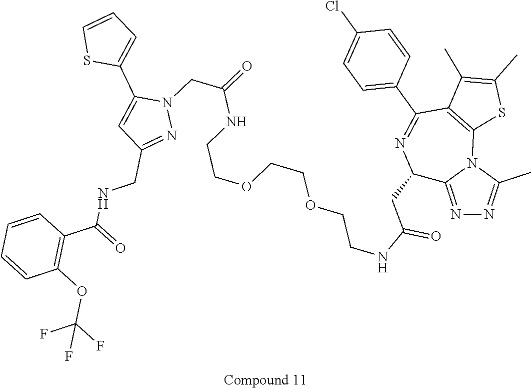 [SMILES: Cc1sc2c(c1C)C(c1ccc(Cl)cc1)=N[C@@H](CC(=O)NCCOCCOCCNC(=O)Cn1nc(CNC(=O)c3ccccc3OC(F)(F)F)cc1-c1cccs1)c1nnc(C)n1-2]

Step 1: Synthesis of N-((1-(tetrahydro-28-pyran-2-yl)-5-(thiophen-2-yl)-1H-pyrazol-3-yl)methyl)-2-(trifluoromethoxy)benzamide.

To a seal tube containing N-((5-iodo-1-(tetrahydro-2H-pyran-2-yl)-1H-pyrazol-3-yl)methyl)-2-(trifluoro methoxy)benzamide (1.4 g, 2.826 mmol) in 1,4-dioxane (16 mL) and water (4 mL) were added thiophen-2-ylboronic acid (0.398 g, 3.106 mmol), NazCO₃ (0.749 g, 7.065 mmol) and Pd(PPh₃)₄ (0.326 g, 0.282 mmol) under argon atmosphere. Then the reaction mixture was stirred in a pre-heated oil bath at 100° C. for 12 h. The mixture was cooled, then diluted with water and extracted with EtOAc trice. The combined organic layer was dried over Na₂SO₄, filtered and concentrated under reduced pressure. The residue was subjected to silica gel column chromatography, eluent: 0-15% EtOAc in petroleum ether to afford the N-((1-(tetrahydro-2H-pyran-2-yl)-5-(thiophen-2-yl)-1H-pyrazol-3-yl)methyl)-2-(trifluoromethoxy)benzamide as an off-white solid (0.434 g, 34.1%). ¹H NMR (400 MHZ, DMSO-d₆) δ 8.94 (t, J=6.0 Hz, 1H), 7.71 (dd, J=5.2, 0.8 Hz, 1H), 7.64-7.54 (m, 2H), 7.50-7.39 (m, 2H), 7.58 (dd, J=3.6, 0.8 Hz, 1H), 7.25-7.17 (m, 1H), 6.40 (s, 1H), 5.33 (dd, J=10.0, 2.0 Hz, 1H), 4.48-4.34 (m, 2H), 3.96 (d, J=12.0 Hz, 1H), 3.70-3.55 (m, 1H), 2.43-2.38 (m, 1H), 2.02-1.90 (m, 1H), 1.89-78 (m, 1H), 1.72-1.57 (m, 1H), 1.56-1.47 (m, 2H).

Step 2: Synthesis of N-((5-(thiophen-2-yl)-1H-pyrazol-3-yl)methyl)-2-(trifluoromethoxy)benzamide.

To a stirred solution of N-((1-(tetrahydro-2H-pyran-2-yl)-5-(thiophen-2-yl)-1H-pyrazol-3-yl)methyl)-2-(trifluoromethoxy)benzamide (570 mg, 1.262 mmol) in DCM (10 mL) and 1,4-dioxane (10 mL) was added 4M HCl in dioxane (15 mL) at 0° C. Then the reaction mixture stirred at room temperature for 12 h. The reaction mixture concentrated under reduced pressure, the residue was basified with saturated NaHCO₃ solution, the solid precipitated was collected by filtration and washed with Et₂O. The solid was then dried to afford the (N-((5-(thiophen-2-yl)-1H-pyrazol-3-yl)methyl)-2-(trifluoromethoxy)benzamide as an off-white solid (434 mg, 93.5%). ¹H NMR (400 MHZ, DMSO-d₆) δ 12.70 (brs, 1H), 8.95 (s, 1H), 7.70-7.55 (m, 2H), 7.53-7.39 (m, 3H), 7.32 (s, 1H), 7.07 (s, 1H), 6.43 (s, 1H), 4.44 (d, J=5.2 Hz, 2H); LC-MS: m/z 386.14 (M+H)⁺.

Step 3: Synthesis of ethyl 2-(5-(thiophen-2-yl)-3-((2-(trifluoromethoxy)benzamido)methyl)-1H-pyrazol-1-yl)acetate.

To a stirred solution of N-((5-(thiophen-2-yl)-1H-pyrazol-3-yl)methyl)-2-(trifluoromethoxy)benzamide (433 mg, 1.178 mmol) in acetonitrile (15 mL) were added K₂CO₃ (244 mg, 1.767 mmol) and ethyl 2-bromoacetate (0.15 mL, 1.414 mmol) at 0° C. Then the reaction mixture was heated and stirred at 80° C. for 12 h, then cooled to room temperature, diluted with water and extracted with EtOAc thrice. The combined organic layer was dried over Na₂SO₄, filtered and concentrated under reduced pressure. The residue was purified by silica gel column chromatography, eluent: 0-20% EtOAc in hexane to afford ethyl 2-(5-(thiophen-2-yl)-3-((2-(trifluoromethoxy)benzamido)methyl)-1H-pyrazol-1-yl)acetate (80 mg, 14.9%) as an off-white solid. LC-MS: m/z 454.1 (M+H)⁺.

Step 4: Synthesis of 2-(5-(thiophen-2-yl)-3-((2-(trifluoromethoxy)benzamido)methyl)-1H-pyrazol-1-yl)acetic acid:

To a stirred solution of ethyl 2-(5-(thiophen-2-yl)-3-((2-(trifluoromethoxy)benzamido)methyl)-1H-pyrazol-1-yl)acetate (90 mg, 0.198 mmol) in THF (2 mL) and water (1 mL) was added LiOH·H₂O (21 mg, 0.496 mmol) at 0° C. Then the reaction mixture was warmed up and stirred at room temperature for 12h. It was concentrated, the residue was acidified with citric acid (PH˜2) and extracted with 10% MeOH in DCM. The combined organic layer was dried over Na₂SO₄, filtered and concentrated under reduced pressure to afford the 2-(5-(thiophen-2-yl)-3-((2-(trifluoromethoxy)benzamido)methyl)-1H-pyrazol-1-yl)acetic acid as an off-white solid (40 mg, 45.8%). ¹H NMR (400 MHZ, DMSO-d₆) δ 13.20 (s, 1H), 8.94 (t, J=5.6 Hz, 1H), 7.68 (d, J=9.2 Hz, 1H), 7.64-7.53 (m, 2H), 7.50-7.39 (m, 2H), 7.22 (d, J=7.6 Hz, 1H), 7.20-7.12 (m, 1H), 6.39 (s, 1H), 4.96 (s, 2H), 4.40 (t, J=5.6 Hz, 2H). LC-MS: 426.1, m/z=(M+H)⁺.

Step 5: Synthesis of(S)-N-((1-(1-(4-(4-chlorophenyl)-2,3,9-trimethyl-6H-thieno[3,2-f][1,2,4]triazolo[4,3-a][1,4]diazepin-6-yl)-2,13-dioxo-6,9-dioxa-3,12-diazapentadecan-15-yl)-S-(thiophen-2-yl)-1H-pyrazol-3-yl)methyl)-2-(trifluoromethoxy)benzamide.

To a stirred solution of(S)—N-(2-(2-(2-aminoethoxy) ethoxy)ethyl)-2-(4-(4-chlorophenyl)-2,3,9-trimethyl-6H-thieno[3,2-f][1,2,4]triazolo[4,3-a][1,4]diazepin-6-yl) acetamide (44 mg, 0.077 mmol) in DMF (2.5 mL) were added DIPEA (23 mg, 0.176 mmol), HATU (30 mg, 0.077 mmol) and 2-(5-(thiophen-2-yl)-3-((2-(trifluoromethoxy)benzamido)methyl)-1H-pyrazol-1-yl)acetic acid (40 mg, 0.070 mmol) at 0° C. Then the reaction mixture was warmed up and stirred at room temperature for 16 h. The reaction mixture was diluted with ice-cold water and extracted with EtOAc thrice. The combined organic layer was dried over Na₂SO₄, filtered and concentrated under reduced pressure The crude was purified by silica gel column chromatography, eluent: 0-4% methanol in DCM to afford(S)-N-((1-(1-(4-(4-chlorophenyl)-2,3,9-trimethyl-6H-thieno[3,2-f][1,2,4]triazolo[4,3-a][1,4]diazepin-6-yl)-2,13-dioxo-6,9-dioxa-3,12-diazapentadecan-15-yl)-5-(thiophen-2-yl)-1H-pyrazol-3-yl)methyl)-2-(trifluoromethoxy)benzamide as a pale yellow gummy solid (33 mg, 37.8% yield). ¹H NMR (400 MHZ, DMSO-d₆) δ 8.93 (t, J=5.6 Hz, 1H), 8.32-8.20 (m, 2H), 7.6 (d, J=4.8 Hz, 1H), 7.63-7.53 (m, 2H), 7.50-7.35 (m, 6H), 7.29-7.24 (m, 1H), 7.18-7.10 (m, 1H), 6.35 (s, 1H), 4.79 (s, 2H), 4.53-4.45 (m, 1H), 4.38 (t, J=6.0 Hz, 2H), 3.53 (s, 4H), 3.49-3.39 (m, 5H), 3.30-3.20 (m, 8H), 2.58 (s, 3H), 2.39 (s, 3H). LC-MS: m/z 938.49 (M+H)⁺.

### Synthesis of(S)-N-(S-amino-1-(thiazolo[4,5-c]quinolin-2-yl)pentyl)-2-(difluoromethoxy)benzamide (Compound 12)

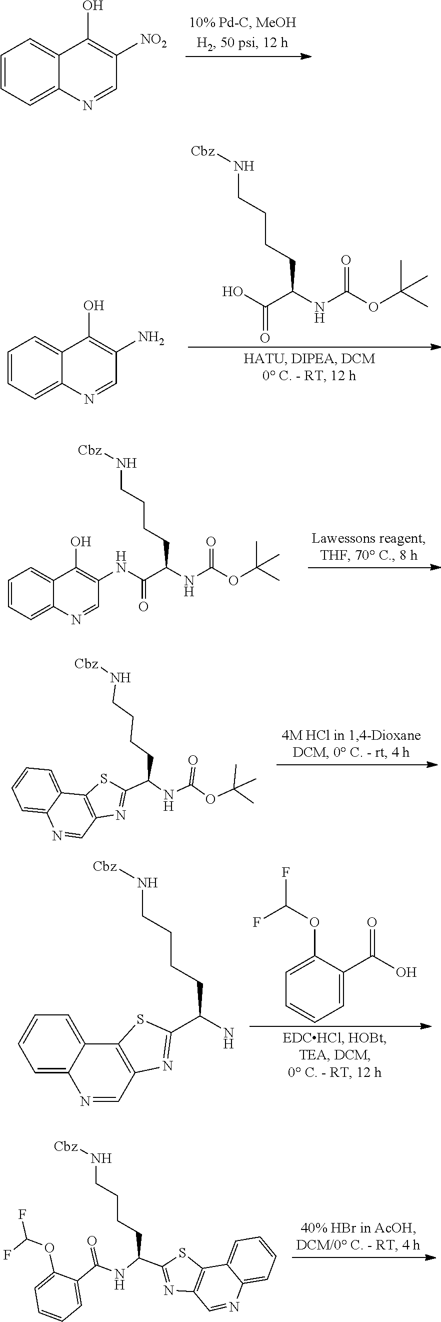

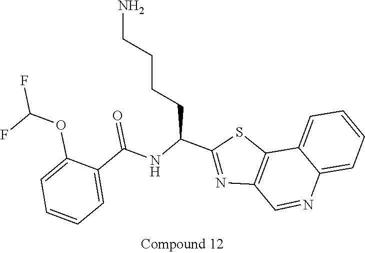 [SMILES: NCCCC[C@H](NC(=O)c1ccccc1OC(F)F)c1nc2cnc3ccccc3c2s1]

Step-1: Synthesis of 3-aminoquinolin-4-ol.

To a stirred solution of 3-nitroquinolin-4-ol (10 g, 3.125 mmol) in MeOH (10 mL) was added 10% Pd/C (5 g) under nitrogen atmosphere at room temperature. The reaction flask was then pressurized with hydrogen gas (50 psi) and the mixture was stirred for 12 h. The resultant reaction mixture was filtered over the celite and the filtrate was concentrated under reduced pressure. The crude product obtained was purified by column chromatography (eluent: 50% EtOAc in hexane) to afford 3-aminoquinolin-4-ol as an off-white solid (7.70 g, 90.53%). LC-MS C₉H₈N₂O m/z (M+H): 161.1.

Step-2: Synthesis of benzyl tert-butyl (6-((4-hydroxyquinolin-3-yl)amino)-6-oxohexane-1,5-diyl) (R)-dicarbamate.

To a stirred solution of 3-Amino 4-hydroxyquinoline (2.50 g, 3.125 mmol) and N6-((benzyloxy) carbonyl)-N2-(tert-butoxycarbonyl)-D-lysine (7.12 g, 3.75 mmol) in DCM at 0° C. was added HATU (7.12 g, 3.75 mmol) followed by DIPEA (8 mL, 9.375 mmol). Then the reaction mixture was stirred at room temperature for 12 h and concentrated. The residue was diluted with H₂O (30 mL) and extracted with EtOAc (2×30 mL). The combined organic layer was washed once with brine solution (20 mL), organic layer was separated, dried over anhydrous Na₂SO₄ concentrated under reduced pressure. The crude product obtained was purified by column chromatography (eluent: 40% EtOAc in Hexane) to afford benzyl tert-butyl (6-((4-hydroxyquinolin-3-yl)amino)-6-oxohexane-1,5-diyl) (R)-dicarbamate as an off-white solid (3.0 g, 88.23%). LC-MS C₂₈H₃₄N₄O₆ m/z (M+H): 523.1.

Step-3: Synthesis of benzyl tert-butyl (1-(thiazolo[4,5-c]quinolin-2-yl) pentane-1,5-diyl) (R)-dicarbamate,

To a stirred solution of benzyl tert-butyl (1-(thiazolo[4,5-c]quinolin-2-yl) pentane-1,5-diyl) (R)-dicarbamate (3.0 g, 5.747 mmol) in THF was added Lawson's reagent (1.85 g, 4.59 mmol) at room temperature. Then the reaction mixture stirred at 70° C. for 8 h and the reaction was quenched with aqueous saturated NH₄Cl solution. It was further diluted with H₂O (30 mL) and extracted with EtOAc (2×30 mL). The combined organic layer was washed once with brine (20 mL), the organic layer was separated, dried over anhydrous Na₂SO₄, and concentrated. The crude compound obtained was purified by column chromatography (eluent: 60% EtOAc in Hexane) to afford benzyl tert-butyl (1-(thiazolo[4,5-c]quinolin-2-yl)pentane-1,5-diyl) (R)-dicarbamate as light-yellow solid (2.2 g, 73.48%). LC-MS: m/z 521.1 (M+H)⁺.

Step-4: Synthesis of benzyl(R)-(5-amino-5-(thiazolo[4,5-c]quinolin-2-yl) pentyl)carbamate:

To a stirred solution benzyl tert-butyl (1-(thiazolo[4,5-c]quinolin-2-yl)pentane-1,5-diyl) (R)-dicarbamate (1.0 g, 1.919 mmol) in DCM (25 mL) at 0° C. was added TFA (5 mL) and the reaction was stirred at room temperature for 4 h. After completion of reaction, solvent was distilled off, then diluted with H₂O (20 mL), basified with aqueous saturated NaHCO₃ (10 mL) and extracted with EtOAc (2×30 mL). The combined organic layer was washed once with brine (20 mL), the organic layer was separated, dried over anhydrous Na₂SO₄ and concentrated. The crude product was used in the next step without further purification (620 mg, 76.78%). LC-MS: m/z 421.1 (M+H)⁺.

Step-5: Synthesis of benzyl(S)-(5-(2-(difluoromethoxy)benzamido)-5-(thiazolo[4,5-c]quinolin-2-yl)pentyl)carbamate.

To a stirred solution of benzyl(R)-(5-amino-5-(thiazolo[4,5-c]quinolin-2-yl) pentyl)carbamate (620 mg 1.4726 mmol) and 2-(difluoromethoxy)benzoic acid (309 mg, 1.6456 mmol) in DCM (20 mL) at 0° C. was added EDC·HCl (428 mg, 2.244 mmol), HOBt (302 mg 2.244 mmol) and TEA (1.4 mL, 7.48 mmol). Then the reaction mixture was stirred at room temperature for 12 h and concentrated. The residue was treated with aqueous NH₄Cl solution and water (20 mL) and extracted with EtOAc (2×20 mL). The combined organic layer was washed once with brine (20 mL), the organic layer was separated, dried over anhydrous Na₂SO₄ and concentrated. The crude product obtained was purified by column chromatography (eluent: 5% MeOH in DCM) to afford benzyl(S)-(5-(2-(difluoromethoxy)benzamido)-5-(thiazolo[4,5-c]quinolin-2-yl)pentyl)carbamate as an off-white solid (350 mg, 40.32%). ¹H NMR (400 MHZ, DMSO) δ 9.43 (s, 1H), 8.45 (d, J=7.2 Hz, 1H), 8.27 (t, J=5.6, Hz 1H), 8.20 (d, J=8.4 Hz, 2H), 7.83 (t, J=7.6 Hz, 1H), 7.75 (t, J=7.6 Hz, 1H), 7.50-7.44 (m, 2H), 7.39-7.30 (m, 3H), 7.26-7.20 (m, 5H), 5.10-5.04 (m, 3H), 3.25-3.21 (m, 2H), 2015-2.11 (m, 1H), 1.96-1.94 (m, 1H), 1.549 (m, 4H). LC-MS: m/z 591.0 (M+H)⁺.

Step-6: Synthesis of(S)—N-(5-amino-1-(thiazolo[4,5-c]quinolin-2-yl)pentyl)-2-(difluoromethoxy)benzamide:

To a stirred solution of benzyl(S)-(5-(2-(difluoromethoxy)benzamido)-5-(thiazolo[4,5-c]quinolin-2-yl)pentyl)carbamate (50 mg, 0.084 mmol) in DCM (5 mL) at 0° C. was added 40% HBr in AcOH and the reaction was stirred at room temperature for 4h. It was then concentrated and the crude product obtained was quenched with saturated NaHCO₃ solution (10 mL) and extracted with EtOAc (2×10 mL). The combined organic layer washed with brine (10 mL), the organic layer was dried over anhydrous Na₂SO₄, and concentrated under reduced pressure. The obtained crude product was purified by prep-HPLC (buffer solution formic acid) to afford(S)—N-(5-amino-1-(thiazolo[4,5-c]quinolin-2-yl)pentyl)-2-(difluoromethoxy)benzamide was obtained as green color sticky compound (5 mg, 38.64%). ¹H NMR (400 MHZ, DMSO) δ 9.39 (s, 1H), 8.26-8.17 (m, 3H), 7.83-7.72 (m, 2H), 7.52-7.42 (m, 2H), 7.30-6.93 (m, 3H), 4.31 (m, 1H), 3.3-3.23 (m, 3H), 2.16-1.766 (m, 2H), 1.54 (m, 5H); LC-MS: m/z 457.29 (M+H)⁺.

### Synthesis of N-(5-amino-1-(5-thioxo-5,6-dihydro-[1,2,4]triazolo[1,5-c]quinazolin-2-yl)pentyl)-2-(difluoro methoxy)benzamide (Compound 13)

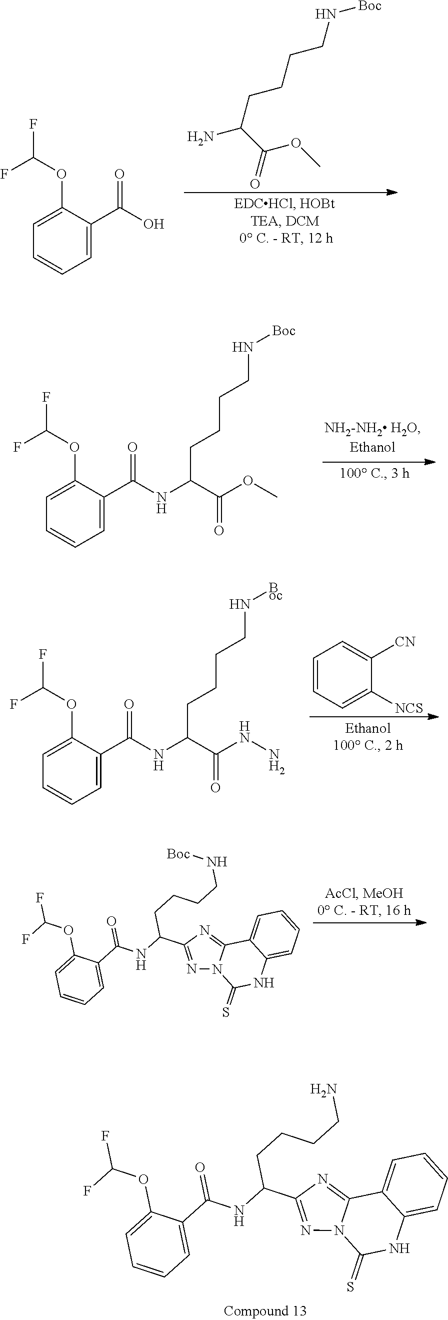

Step-1: Synthesis of methyl N6-(tert-butoxycarbonyl)-N2-(2-(difluoromethoxy)benzoyl) lysinate.

To a stirred solution of methyl N6-(tert-butoxycarbonyl) lysinate (1.70 g, 5.851 mmol) and 2-(difluoromethoxy)benzoic acid (1.20 g, 6.43 mmol) in DCM (15 mL) at 0° C. were added EDC·HCl (1.84 gm, 9.64 mmol), HOBt (1.30 gm 9.64 mmol), followed by TEA (4 mL, 29.250 mmol). Then the reaction mixture was stirred at room temperature for 12 h and concentrated. The residue was diluted with H₂O (20 mL) and extracted with EtOAc (2×20 mL). The combined organic layer was washed once with saturated brine (20 mL), the organic layer was separated, dried over anhydrous Na₂SO₄ and concentrated. The crude product obtained was purified by column chromatography (eluent: 5% MeOH in DCM) to afford methyl N6-(tert-butoxycarbonyl)-N2-(2-(difluoromethoxy)benzoyl) lysinate as an off white solid (1.10 g, 40.32%). LC-MS: m/z (M+H): 331.1.

Step-2: Synthesis of tert-butyl (5-(2-(difluoromethoxy)benzamido)-6-hydrazinyl-6-oxohexyl)carbamate:

To a stirred solution of methyl N6-(tert-butoxycarbonyl)-N2-(2-(difluoromethoxy)benzoyl) lysinate (500 mg, 1.62 mmol) in EtOH (5 mL) was added aqueous hydrazine solution (1 mL) and reaction mixture stirred at room temperature for 15 min and at 100° C. for 3 h. The reaction mixture was then concentrated and the residue was treated with H₂O (10 mL) and extracted with EtOAc (2×20 mL). The combined organic layer was washed once with brine (20 mL), the organic layer was separated, dried over anhydrous Na₂SO₄ and concentrated. The crude product obtained was purified by column chromatography (20% EtOAc in hexane) to tert-butyl (5-(2-(difluoromethoxy)benzamido)-6-hydrazinyl-6-oxohexyl)carbamate was obtained as dark brown color liquid (200 mg, 40%). LC-MS: m/z 431.10 (M+H).

Step-3: Synthesis of tert-butyl (5-(2-(difluoromethoxy)benzamido)-5-(5-thioxo-5,6-dihydro-[1,2,4]triazolo[1,5-c]quinazolin-2-yl)pentyl)carbamate.

To a stirred solution of tert-butyl (5-(2-(difluoromethoxy)benzamido)-6-hydrazinyl-6-oxohexyl)carbamate (120 mg, 0.27 mmol) in EtOH (10 mL) was added 2-isothiocyanatobenzonitrile (49 mg, 0.30 mmol) and reaction mixture stirred at room temperature for 15 min and then at 100° C. for 2 h. The reaction mixture was then concentrated, diluted with H₂O (10 mL) and extracted with EtOAc (2×20 mL). The combined organic layer was washed once with brine (20 mL), the organic layer was separated, dried over anhydrous Na₂SO₄ and concentrated. The crude obtained was purified by prep-HPLC (buffer solution NH₄OAc, MeOH) to obtain tert-butyl (5-(2-(difluoromethoxy)benzamido)-5-(5-thioxo-5,6-dihydro-[1,2,4]triazolo[1,5-c]quinazolin-2-yl)pentyl)carbamate was obtained as white solid (10 mg, 6.28%). ¹H NMR (400 MHZ, DMSO) δ 14.01 (s, 1H), 8.91 (d, J=8.4, 1H), 8.20 (d, J=7.6, 1H), 7.81-7.77 (m, 1H), 7.67-7.50 (m, 4H), 7.40-6.79 (m, 4H), 5.99-5.71 (m, 1H), 2.92-2.91 (m, 2H), 2.00-1.91 (m, 2H), 1.44 (m, 4H), 1.34 (s, 9H); LC-MS: m/z 571.10 (M−H).

Step-4: N-(5-amino-1-(5-thioxo-5,6-dihydro-[1,2,4]triazolo[1,5-c]quinazolin-2-yl)pentyl)-2-(difluoro methoxy)benzamide.

To a stirred solution of tert-butyl (5-(2-(difluoromethoxy)benzamido)-5-(5-thioxo-5,6-dihydro-[1,2,4]triazolo[1,5-c]quinazolin-2-yl)pentyl)carbamate (50 mg, 0.087 mmol) in methanol (5 mL) at 0° C. was added acetyl chloride (0.1 mL) and reaction stirred at room temperature for 16 h. The reaction mixture was then concentrated and the crude product was purified by prep-HPLC (buffer solution NH₄OAc, MeOH) to afford N-(5-amino-1-(5-thioxo-5,6-dihydro-[1,2,4]triazolo[1,5-c]quinazolin-2-yl)pentyl)-2-(difluoromethoxy)benzamide as pale-yellow solid (20 mg, 48.48%). ¹H NMR (400 MHZ, DMSO) δ 14.05 (s, 1H), 8.95 (d, J=8.4 Hz, 1H), 8.19 (d, J=7.6 Hz, 1H), 7.80 (t, J=8.4 Hz, 1H), 7.74 (s, 2H), 7.68 (d, J=8.0 Hz, 1H), 7.59-7.52 (m, 3H), 7.39-7.02 (m, 3H), 5.33 (q, J=8.8 Hz, 1H), 2.82-2.75 (m, 2H), 2.10-1.92 (m, 2H), 1.63-1.47 (m, 4H); LC-MS: m/z 473.38 (M+H).

### Synthesis of (R)-N-(S-amino-1-(5-(2-methoxyphenyl)-1H-pyrazol-3-yl) pentyl)-2-(difluoromethoxy)benzamide (Compound 14)

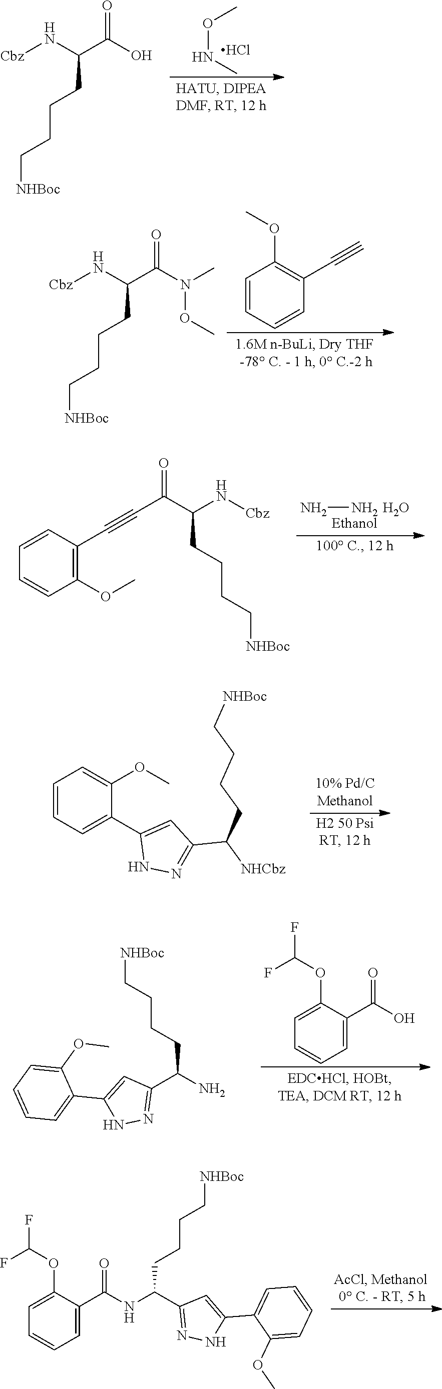

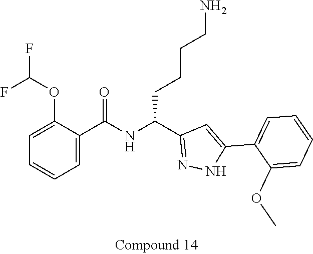 [SMILES: COc1ccccc1-c1cc([C@@H](CCCCN)NC(=O)c2ccccc2OC(F)F)n[nH]1]

Step 1: Synthesis of Benzyl tert-butyl (6-(methoxy(methyl)amino)-6-oxohexane-1,5-diyl) (R)-dicarbamate.

To a stirred solution of (R)-N-benzyloxycarbonyl-N6-(tert-butoxycarbonyl)-L-lysine (1 g, 26.29 mmol) and HATU (1.5 g, 39.43 mmol) in DMF (10 mL) were added N, O-dimethyl hydroxylamine hydrochloride (260 mg, 26.29 mmol) and DIPEA (1 g, 78.86 mmol). Then the reaction mixture was stirred at room temperature for 12 h. After completion of reaction, it was diluted with water (10 mL) and extracted with EtOAc (3×50 mL). The combined organic layer was concentrated. The crude compound was purified by flash column chromatography (eluent: 20% ethyl acetate in hexane) to afford benzyl tert-butyl (6-(methoxy(methyl)amino)-6-oxohexane-1,5-diyl) (R)-dicarbamate (600 mg, 54.6%) as a colorless viscous liquid. LC-MS m/z (M+H-Boc): 324.1.

Step 2: Synthesis of benzyl tert-butyl (8-(2-methoxyphenyl)-6-oxooct-7-yne-1,5-diyl) (S)-dicarbamate.

To a stirred solution of 1-ethynyl-2-methoxybenzene (100 mg, 0.2361 mmol) in dry THF (2 mL) at −78° C. was added n-BuLi in THF (0.6 mL, 0.9445 mmol) over 10 minutes. The reaction mixture was stirred at −78° C. for 1h followed by the dropwise addition of benzyl tert-butyl (6-(methoxy(methyl)amino)-6-oxohexane-1,5-diyl) (R)-dicarbamate (from step 1) in THE over 10 min. Then the reaction mixture was warmed up and stirred at 0° C. for 2 h. It was then quenched by the addition of aqueous ammonium chloride solution (10 mL) and extracted with EtOAc (2×50 mL). The combined organic layer was dried over Na₂SO₄ and concentrated. The crude compound was purified by flash column chromatography (eluent: 20% EtOAc in hexane) to afford benzyl tert-butyl (8-(2-methoxyphenyl)-6-oxooct-7-yne-1,5-diyl) (S)-dicarbamate (60 mg, 51.4%) as a colorless viscous liquid. ¹H NMR (400 MHZ, DMSO) δ 7.51-7.43 (m, 2H), 7.37-7.29 (m, 5H), 6.97-6.89 (m, 2H), 5.66 (d, J=7.2 Hz, 1H), 5.13 (s, 2H), 4.59-4.57 (m, 2H), 3.86 (s, 3H), 3.12 (q, J=6.4 Hz, 2H), 2.12-2.07 (m, 1H), 1.87-1.81 (m, 1H), 1.54-1.51 (m, 2H), 1.41 (s, 9H); LC-MS m/z (M+H-Boc): 395.1.

Step 3: Synthesis of benzyl tert-butyl (1-(5-(2-methoxyphenyl)-1H-pyrazol-3-yl)pentane-1,5-diyl) (R)-dicarbamate.

To a stirred solution of benzyl tert-butyl (8-(2-methoxyphenyl)-6-oxooct-7-yne-1,5-diyl) (S)-dicarbamate (60 mg, 0.1213 mmol) in ethanol (1 mL) was added hydrazine hydrate (15.5 mg, 0.4852 mmol). The resulting solution was heated at 100° C. for 12 h and concentrated. To the residue, water (10 mL) was added, and the mixture was extracted with EtOAc (2×50 mL). The combined organic layer was dried over Na₂SO₄ and concentrated. The crude compound was purified by flash column chromatography (eluent: 70-80% EtOAc in hexane) to afford benzyl tert-butyl (1-(5-(2-methoxyphenyl)-1H-pyrazol-3-yl)pentane-1,5-diyl) (R)-dicarbamate (50 mg, 86.6% Yield) as white sticky liquid. ¹H NMR (400 MHZ, DMSO) δ 12.60 (bs, 1H), 7.61 (bs, 2H), 7.36-7.30 (m, 6H), 7.11 (d, J=7.6 Hz, 1H), 6.99 (t, J=7.2 Hz, 1H), 6.77 (t, J=5.6 Hz, 1H), 6.56 (s, 1H), 5.03 (s, 2H), 4.62 (d, J=8.0 Hz, 1H), 3.86 (s, 3H), 2.88 (q, J=6.4 Hz, 2H), 1.73-1.68 (m, 2H), 1.35 (s, 9H), 1.23-1.15 (m, 4H). LC-MS m/z (M+H): 509.11.

Step 4: Synthesis of tert-butyl(R)-(5-amino-5-(5-(2-methoxyphenyl)-1H-pyrazol-3-yl)pentyl)carbamate.

To a stirred solution of benzyl tert-butyl (1-(5-(2-methoxyphenyl)-1H-pyrazol-3-yl)pentane-1,5-diyl) (R)-dicarbamate (100 mg, 0.2103 mmol) in methanol (3 mL) at room temperature was added 10% Pd/C. Then the reaction was stirred at room temperature in the presence of hydrogen atmosphere for 12 h. After completion of reaction, the reaction mixture was filtered through pad of celite and washed with methanol (2×20 mL). The combined filtrate was concentrated on rotavapor to get the desired compound tert-butyl(R)-(5-amino-5-(5-(2-methoxyphenyl)-1H-pyrazol-3-yl)pentyl)carbamate (52 mg, 66.12% Yield) as white solid. ¹H NMR (400 MHZ, DMSO) δ 12.59 (bs, 1H), 7.74 (bs, 1H), 7.30 (t, J=7.2 Hz, 1H), 7.11 (d, J=8.4 Hz, 1H), 6.99 (t, J=8 Hz, 1H), 6.76 (t, J=5.2 Hz, 1H), 6.61 (s, 1H), 3.91 (t, J=6.4 Hz, 1H), 3.87 (s, 3H), 2.88 (q, J=6.4 Hz, 2H), 1.68-1.66 (m, 2H), 1.35 (s, 9H), 1.35-1.31 (m, 4H); LC-MS m/z (M+H): 375.1.

Step 5: Synthesis of tert-butyl(R)-(5-(2-(difluoromethoxy)benzamido)-5-(5-(2-methoxyphenyl)-1H-pyrazol-3-yl)pentyl)carbamate.

To a stirred solution of tert-butyl(R)-(5-amino-5-(5-(2-methoxyphenyl)-1H-pyrazol-3-yl)pentyl)carbamate (50 mg, 0.1337 mmol), 2-(difluoromethoxy)benzoic acid (25 mg, 0.1337 mmol), EDC·HCl (38.5 mg, 0.2006 mmol), and HOBt (27 mg, 0.2006 mmol) in DCM (2 mL) was added TEA (41 mg, 0.4012 mmol) at room temperature. Then the reaction mixture was stirred at room temperature for 12 h and concentrated. The residue was then diluted with water (10 mL) and extracted with EtOAc (2×50 mL). The combined EtOAc layer was dried over sodium sulfate and concentrated. The crude compound was purified by flash column chromatography (eluent: 2% MeOH+DCM) to afford tert-butyl(R)-(5-(2-(difluoromethoxy)benzamido)-5-(5-(2-methoxyphenyl)-1H-pyrazol-3-yl)pentyl)carbamate (45 mg, 61.88%) as off-white solid. LC-MS m/z (M+H): 545.1.

Step 6: Synthesis of (R)-N-(S-amino-1-(5-(2-methoxy phenyl)-1H-pyrazol-3-yl)pentyl)-2-(difluoro methoxy)benzamide.

To a stirred solution of tert-butyl(R)-(5-(2-(difluoromethoxy)benzamido)-5-(5-(2-methoxyphenyl)-1H-pyrazol-3-yl)pentyl)carbamate (40 mg, 0.0735 mmol) in MeOH (2 mL) at 0° C. was added acetyl chloride (2 mL) dropwise. Then the reaction mixture was stirred at room temperature for 5 h and concentrated. The crude compound obtained was washed by diethyl ether (2×10 mL) and dried on rotavapour. The resultant crude compound was further purified by prep-HPLC to get (R)—N-(5-amino-1-(5-(2-methoxyphenyl)-1H-pyrazol-3-yl)pentyl)-2-(difluoromethoxy)benzamide (14 mg, 42.88%) as off white solid. ¹H NMR (400 MHZ, DMSO) δ 12.69 (bs, 1H), 8.58 (d, J=7.6 Hz, 1H), 7.71 (bs, 1H), 7.55-7.50 (m, 2H), 7.37-6.99 (m, 6H), 6.65 (s, 1H), 5.13 (t, J=8 Hz, 1H), 3.87 (s, 3H), 2.63-2.60 (m, 2H), 1.88-1.74 (m, 2H), 1.41-1.39 (m, 4H), LC-MS m/z (M+H): 445.1.

The following degrader molecules can be prepared using the amide coupling between the targeting molecule and the E2 binder.

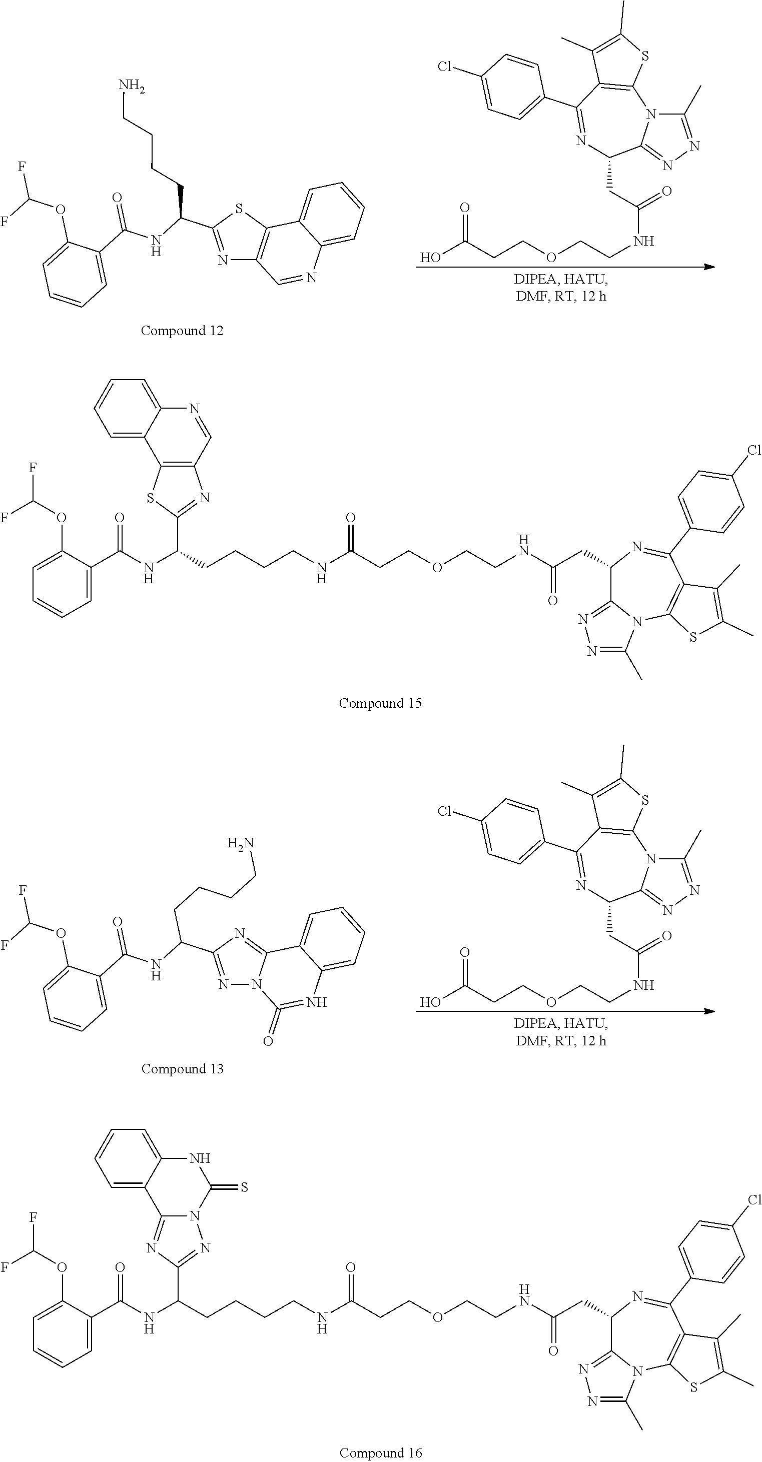

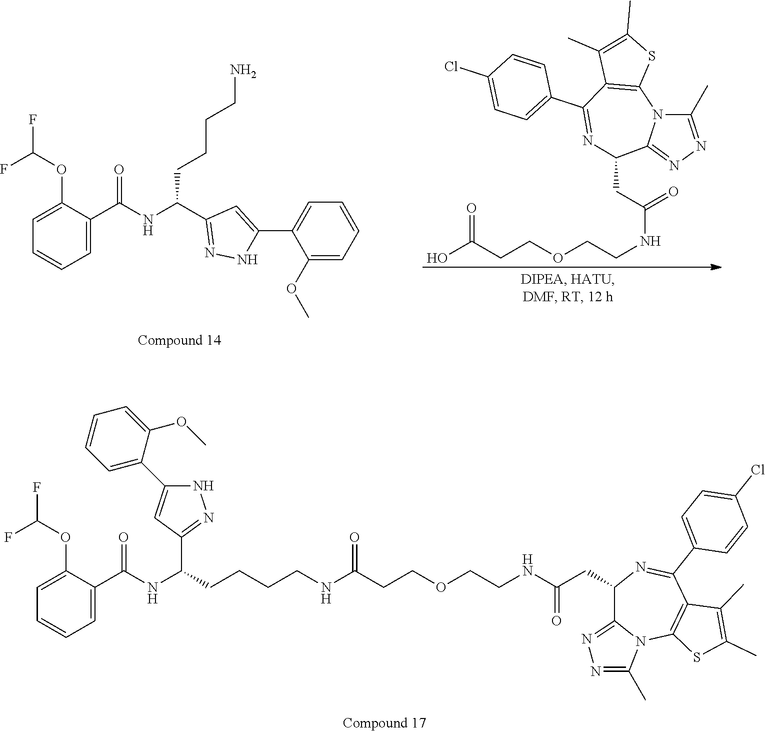 [SMILES: COc1ccccc1-c1cc([C@@H](CCCCN)NC(=O)c2ccccc2OC(F)F)n[nH]1 ; Cc1sc2c(c1C)C(c1ccc(Cl)cc1)=N[C@@H](CC(=O)NCCOCCC(=O)O)c1nnc(C)n1-2 ; COc1ccccc1-c1cc([C@H](CCCCNC(=O)CCOCCNC(=O)C[C@@H]2N=C(c3ccc(Cl)cc3)c3c(sc(C)c3C)-n3c(C)nnc32)NC(=O)c2ccccc2OC(F)F)n[nH]1]

### Biological Assays

### UBE2K Dissociation Constant (Kd) of E2-Binder Compound 1

UBE2K Ligand binding to mono-Ub UBE2K and UBE2K was measured by Surface plasma resonance using a Biacore SPR S200 instrument. The proteins and ligands were prepared for experimental measurement as described below. Avi tagged proteins were biotinylated and were immobilized on a Series S/Sensor Chip SA. Proteins were immobilized based on their molar weight as follows-Biotinylated Avi peptide (1000 μM), Biotinylated UBE2K-Ub Complex (12 μM) and Biotinylated UBE2K Wild Type (3 μM). Proteins were immobilized using a buffer comprised of 1X HBS-N Buffer+0.05% Tween 20 (250 mL). A stock of Compound 1 was prepared in affinity buffer comprised of 1% DMSO, 1X PBS-P+Buffer. Further dilution of stock were prepared in 0% DMSO, 1X PBS-P+Buffer or 3% DMSO, 1X PBS-P+Buffer and were used as running buffers. Compound 1 was prepared as 2 mM or 750 AM stocks in the buffers described above. Titration was performed using the Liquid handler/TECAN EVO (WALL-E) as follows. 1 or 2, 96 deep well plates (1.2 mL) were used for performing titrations-Aliquoted 3% DMSO, 1X PBS-P+buffer. 1:2 dilution was performed by WALL-E (250 μL+250 μL) for the rest of titrations till 3.12p M. The titrations of Compound 1 was then transferred to 384 deep well plate in duplicates side-by-side (125 μL) each. This plate was then covered with a 384 well S200 compatible foil and used for the affinity method. Solvent Correction buffer with DMSO concentration + and −0.5% DMSO of running buffer was run in each experimental run.

Data was collected and processed in accordance with the methodologies described in the Biacore S200 Evaluation Software. Steady state fitting was performed on the data using the software and data represented as sensogram to deconvolute binding and dissociation constants as described in Dahl,G et al., SLAS Discovery, 2017, Vol. 22 (2) 203-209. Results produced a Kd(μM) of 2.11 for Compound 1. This data established that Compound 1 is a useful UBE2K binder for subsequent PROTAC generation.

### Dose Dependent Degradation of BRD4 in Hela Cells

UBE2K binder Compound 1 was used as a precursor to generate PROTAC Compounds 2, 6, 7, and 9. See experimental section above. Each of these PROTACs were first tested for their ability to bind the E2 enzyme UBE2K.

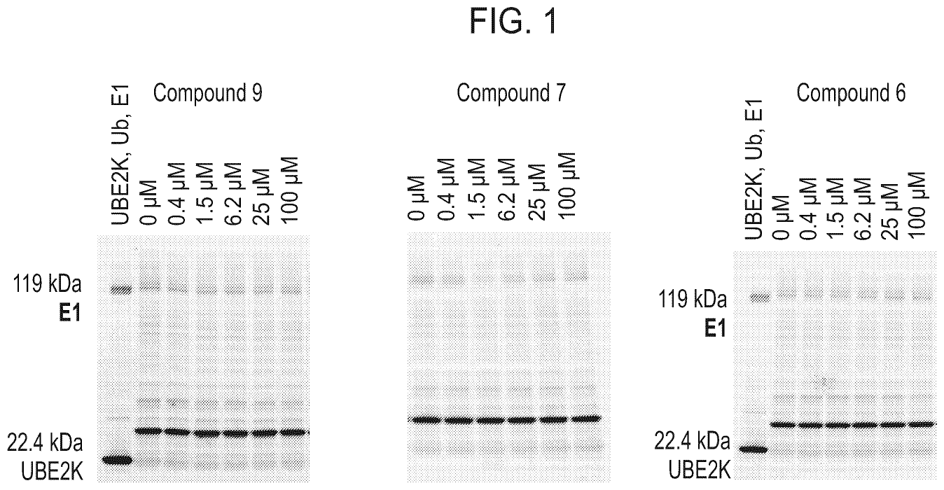

All compounds except for Compound 2 bind UBE2K-Ub. See FIG. 1.

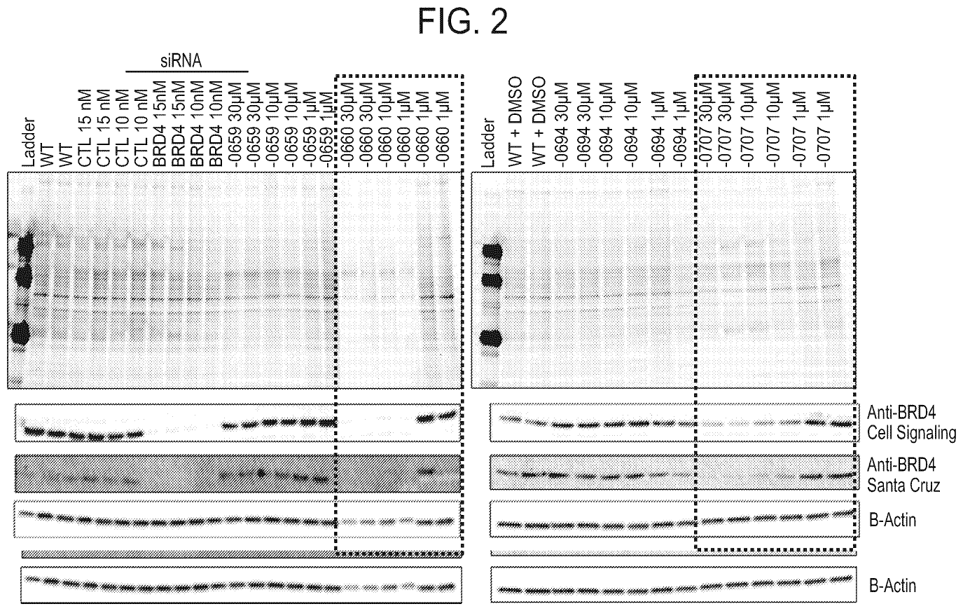

Compounds 6, 7, and 9 were then investigated for their ability to degrade BRD4. As shown by FIG. 2, Compounds 7 and 9 showed concentration dependent loss of BRD4. This data shows that targeting E2, in particular UBE2K, is a viable strategy for generating novel PROTACs with therapeutic relevance.

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound comprising an E2 binding moiety, a target protein binding moiety, and a linker (L) which covalently attaches the E2 binding moiety to the target protein binding moiety.

2. The compound of claim 1, wherein the E2 binding moiety is selected from one that binds an E2 enzyme selected from UBE2A, UBE2B, UBE2C, UBE2D1, UBE2D2, UBE2D3, UBE2D4, UBE2E1, UBE2E2, UBE2E3, UBE2G1, UBE2G2, UBE2H, UBE2J1, UBE2J2, UBE2K, UBE2L3, UBE2N, UBE2NL, UBE20, UBE2Q1, UBE2Q2, UBE2QL, UBE2R¹, UBE2R², UBE2S, UBE2T, UBE2U, UBE2V1, UBE2V2, UBE2W, and BIRC6.

3. The compound of claim 1, wherein the E2 binding moiety is one that binds UBE2K.

4. The compound of claim 1, wherein the E2 binding moiety is a compound of the Formula I: 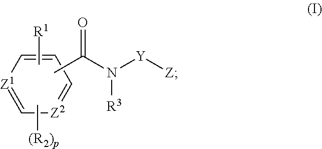
wherein
Z¹ and Z² are each independently N or CH;
R¹ is (C₁-C₆)alkyl, halo(C₁-C₆)alkyl, (C₁-C₆)alkoxy, halo(C₁-C₆)alkoxy, or —NR^{c}R^{d}, wherein two available hydrogen atoms on said halo(C₁-C₆)alkyl and halo(C₁-C₆)alkoxy may be taken together to which the carbon atoms they are attached to form a 3- to 6-membered cycloalkyl optionally substituted with 1 to 3 groups selected from halo, (C₁-C₆)alkyl, halo(C₁-C₆)alkyl, (C₁-C₆)alkoxy, and halo(C₁-C₆)alkoxy;
R² is CN, halo, OH, (C₁-C₆)alkyl, halo(C₁-C₆)alkyl, (C₁-C₆)alkoxy, or halo(C₁-C₆)alkoxy; or R¹ and R², when on adjacent carbon atoms, are taken together with the carbon atoms to which they are attached to form a 5- or 6-membered oxygen containing heterocyclyl optionally substituted with 1 to 3 groups selected from halo, (C₁-C₆)alkyl, and halo(C₁-C₆)alkyl;
R³ is hydrogen, (C₁-C₆)alkyl, or halo(C₁-C₆)alkyl;
Y is CH₂, —CHR^{a}, —CR^{a}R^{b}, S, or SO;
p is 0 or 1;
R^{a} and R^{b} are each independently halo, (C₁-C₆)alkyl, or halo(C₁-C₆)alkyl; or R^{a} and R^{b} together with the carbon atom they are bound for a 3- to 6-membered cycloalkyl or a 3- to 6-membered heterocyclyl, each of which are optionally substituted with 1 to 3 groups selected from halo, (C₁-C₆)alkyl, halo(C₁-C₆)alkyl, (C₁-C₆)alkoxy, halo(C₁-C₆)alkoxy, (C₁-C₆)alkylOH, (C₁-C₆)alkylO(C₁-C₆)alkyl, and OH;
R^{c} and R^{d} are each independently hydrogen (C₁-C₆)alkyl, halo(C₁-C₆)alkyl, (C₁-C₆)alkylO(C₁-C₆)alkyl, halo(C₁-C₆)alkylO(C₁-C₆)alkyl, (C₁-C₆)alkyl-O-halo(C₁-C₆)alkyl, halo(C₁-C₆)alkyl-O-halo(C₁-C₆)alkyl, or (C₁-C₆)alkylOH; or R^{c} and R^{d} together with the nitrogen atom they are bound form a 4- to 7-membered heterocyclyl optionally substituted with 1 to 3 groups selected from halo, (C₁-C₆)alkyl, halo(C₁-C₆)alkyl, (C₁-C₆)alkoxy, halo(C₁-C₆)alkoxy, and oxo;
Z is a tricyclic fused ring having the formula: 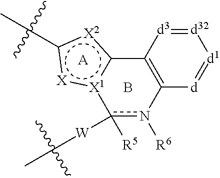
ring A is aromatic;
the wavy bond on ring A denotes the point of attachment to Y;
the wavy bond next to W denotes the point of attachment to Linker (L);
X, X¹, and X² are each, as valency permits, independently selected from —CR⁷, N, O, and S;
the dotted line in ring B represents a single or double bond;
W is NH, —N(C₁-C₆)alkyl), O, or S;
R⁵ is selected from hydrogen, (C₁-C₆)alkyl, halo(C₁-C₆)alkyl, (C₁-C₆)alkoxy, halo(C₁-C₆)alkoxy, —S(C₁-C₆)alkyl, —SH, OH, (C₃-C₆)cycloalkyl, (C₄-C₇)heterocyclyl, and —NR^{e}R^{f}, wherein said (C₃-C₆)cycloalkyl and (C₄-C₇)heterocyclyl are each optionally substituted with 1 to 3 groups selected from halo, (C₁-C₆)alkyl, halo(C₁-C₆)alkyl, (C₁-C₆)alkoxy, halo(C₁-C₆)alkoxy, and oxo;
R⁶ is hydrogen or (C₁-C₆)alkyl when the dotted line in ring B is a single bond, or R⁶ is absent when the dotted line in ring B is a double bond;
d, d₁, d₂ and d₃ are each independently selected from CR⁸ and N;
R^{e} and R^{f} are each independently hydrogen, (C₁-C₄)alkyl, (C₁-C₄)alkylNH (C₁-C₄)alkyl, (C₁-C₄)alkylN [(C₁-C₄)alkyl]₂, (C₁-C₄)alkylO(C₁-C₄)alkyl;
R⁷ is hydrogen or (C₁-C₆)alkyl; and
R⁸ is halogen, hydrogen, (C₁-C₆)alkyl, halo(C₁-C₆)alkyl, CN, or OH.

5. The compound of claim 4, wherein the E2 binding moiety is a compound of the Formula II:

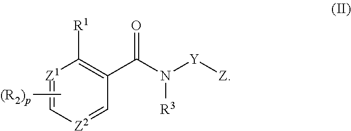

6. The compound of claim 5, wherein
W is S;
R³ is hydrogen;
Z¹ and Z² are each CH;
Y is CH₂;
p is 0;
R¹ is (C₁-C₆)alkyl, halo(C₁-C₆)alkyl, halo(C₁-C₆)alkoxy, (C₁-C₆)alkoxy, or —NR^{c}R^{d}; and
R^{c} and R^{d} together with the nitrogen atom they are bound form a 5- to 6-membered heterocyclyl optionally substituted with 1 to 3 halo;
X is N;
X¹ is CH or N;
X² is CH, N, S, or O;
d is N or —CR⁸;
d₂ is N or —CR⁸;
d¹ is —CR⁸; and
d₃ is —CR⁸.

7-21. (canceled)

22. The compound of claim 6, wherein Z is selected from

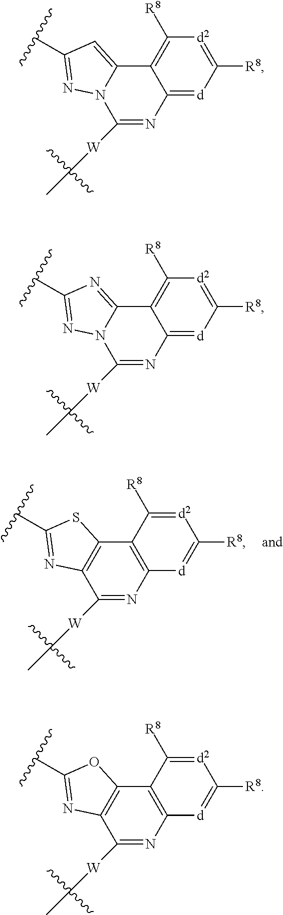

23. The compound of claim 6, wherein Z is selected from

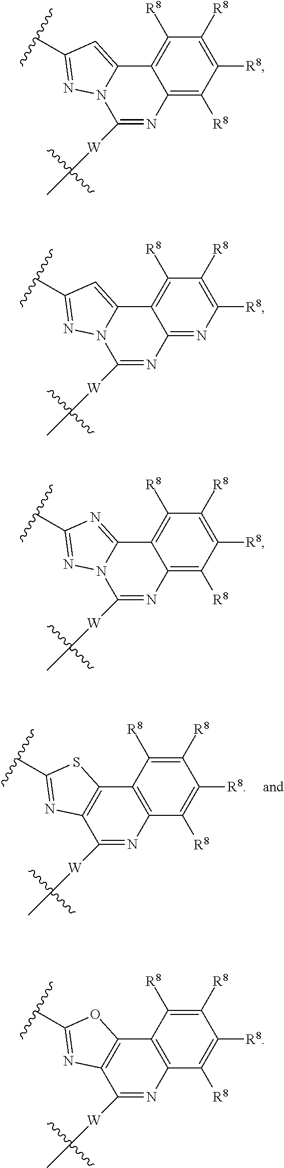

24. The compound of claim 23, wherein
R⁸ is hydrogen or halo;
R⁵ is hydrogen; and
R⁶ is selected from hydrogen and (C₁-C₄)alkyl.

25-28. (canceled)

29. The compound of claim 1, wherein the E2 binding moiety is a compound of the Formula III: 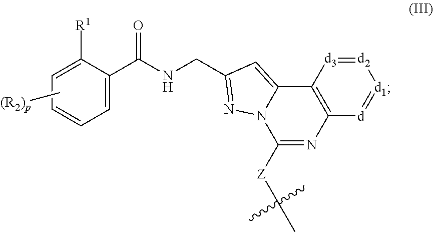
wherein
d, d₁, d₂ and d₃ are each CR⁸;
p is 0; and
Z is S.

30. (canceled)

31. (canceled)

32. The compound of claim 1, wherein the E2 binding moiety is of the structure:

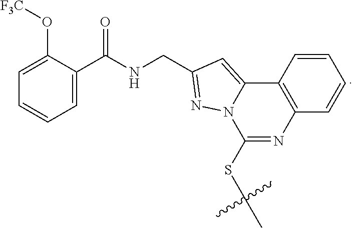 [SMILES: *Sc1nc2ccccc2c2cc(CNC(=O)c3ccccc3OC(F)(F)F)nn12]

33. The compound of claim 1, wherein the target protein binding moiety is one that binds a protein selected from STAT3, BCL2, WRN, and BRD4.

34. (canceled)

35. The compound of claim 1, wherein the target protein binding moiety is of the structure: 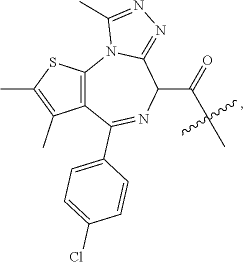 [SMILES: *C(=O)C1N=C(c2ccc(Cl)cc2)c2c(sc(C)c2C)-n2c(C)nnc21]
wherein the wavy bond denotes the point of attachment to Linker (L).

35. The compound of claim 1, wherein linker (L) comprises an optionally substituted straight or branched alkyl group which is optionally interrupted by one or more heteroatoms selected from O, N, and S.

36. (canceled)

37. The compound of claim 1, wherein Linker (L) is of the following structure: 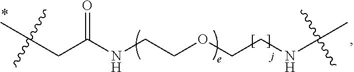
wherein the asterisk (*) denotes the point of attachment to the E2 binding moiety; e is an integer from 0 to 4; and j is an integer from 0 to 6.

38-40. (canceled)

41. The compound of claim 1, wherein the compound is of the structural formula: 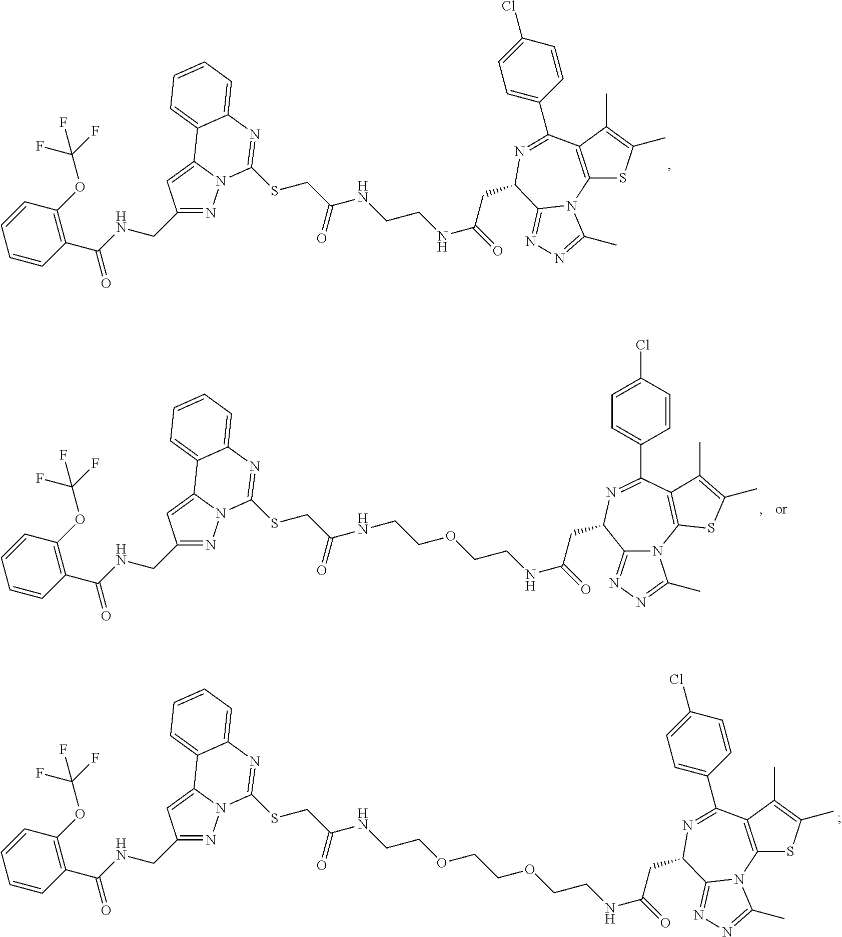 [SMILES: Cc1sc2c(c1C)C(c1ccc(Cl)cc1)=N[C@@H](CC(=O)NCCNC(=O)CSc1nc3ccccc3c3cc(CNC(=O)c4ccccc4OC(F)(F)F)nn13)c1nnc(C)n1-2 ; Cc1sc2c(c1C)C(c1ccc(Cl)cc1)=N[C@@H](CC(=O)NCCOCCNC(=O)CSc1nc3ccccc3c3cc(CNC(=O)c4ccccc4OC(F)(F)F)nn13)c1nnc(C)n1-2 ; Cc1sc2c(c1C)C(c1ccc(Cl)cc1)=N[C@@H](CC(=O)NCCOCCOCCNC(=O)CSc1nc3ccccc3c3cc(CNC(=O)c4ccccc4OC(F)(F)F)nn13)c1nnc(C)n1-2]
or a pharmaceutically acceptable salt thereof.

42. A pharmaceutical composition comprising the compound of claim 1, or a pharmaceutically acceptable salt thereof; and a pharmaceutically acceptable carrier.

43. A method of treating a disease responsive to degradation of a target protein comprising administering to the subject an effective amount of a compound of claim 1.

44. The method of claim 43, wherein the target protein is selected from STAT3, BCL2, WRN, and BRD4.

45. The method of claim 43, wherein the disease is selected from a cancer, an inflammatory disease, sepsis, an autoimmune disease, and a viral infection.
